(19) United States
(12) Patent Application Publication
(10) Pub. No.: US 2004/0029130 A1
(43) Pub. Date: Feb. 12, 2004

(54) Drug screening systems and assays

(76) Inventors: Steve Craig West, South Mimms Hertfordshire (GB); Michael Bartlett-Jones, London (GB); Leslyn Ann Akemi Hanakahi, Baltimore, MD (US)

Correspondence Address: FISH & RICHARDSON PC, 225 FRANKLIN ST, BOSTON, MA, 02110, US

(21) Appl. No.: 10/296,014

(22) PCT Filed: May 18, 2001
(86) PCT No.: PCT/GB01/02180

(30) Foreign Application Priority Data
May 20, 2000 (GB) 0012179.8

(51) Int. Cl. C12Q 1/68; C12P 19/34

(52) U.S. Cl. 435/6; 435/91.2

(57) ABSTRACT

A method of stimulating non-homologous end-joining (NHEJ) of DNA the method comprising performing NHEJ of DNA in the presence of inositol hexakisphosphate (IP₆) or other stimulatory inositol phosphate. An assay of a protein kinase wherein the assay comprises inositol hexakisphosphate (IP₆) or other stimulatory inositol phosphate. The invention also provides screening assays for compounds which may modulate NHEJ and which may be therapeutically useful; and screening assays for compounds which may modulate DNA-PK and related protein kinases and which may be therapeutically useful. Methods of modulating NHEJ and protein kinases are also disclosed.

The present invention relates to assays and drug screening systems involving components of the non-homologous end joining (NHEJ) pathway, and to screening systems which make use of the protein kinase known as DNA-PK (DNA-dependent protein kinase) and related protein kinases such as ATR, ATM and FRAP. The invention also relates to inositol hexakisphosphate (IP₆), inositol pentakisphosphate (IP₅), inositol tetrakisphosphate (IP₄), diphosphoinositol pentakisphosphate (IP₇) and bis-diphosphoinositol tetrakisphosphate (IP₈).

The repair of double strand breaks (DSBs) in DNA is essential for the maintenance of genomic stability. Failure to repair DSBs can result in the loss of genetic information, chromosomal translocations and cell death. Two mechanisms for the repair of DSBs have been described, involving either homologous recombination or non-homologous end-joining (NHEJ). Homologous recombination is particularly effective in S-phase when the break can be repaired using genetic information from a sister chromatid, whereas NHEJ is thought to be effective at all times in the cell cycle (Essers et al, 2000; Takata et al, 1998). NHEJ also plays an important role in DSB repair during V(D)J recombination (Blunt et al, 1995; Taccioli et al, 1993).

The repair of double-strand breaks by non-homologous end joining requires the products of the XRCC4, XRCC5, XRCC6 and XRCC7 genes (reviewed by (Chu, 1997; Critchlow and Jackson, 1998; Weaver, 1996). XRCC4 encodes a protein (XRCC4) that forms a heterodimer with DNA ligase IV, XRCC5 and XRCC6 encode the 70 and 80 kDa subunits of the DNA end-binding protein Ku, and XRCC7 encodes the catalytic subunit of the DNA-dependent protein kinase DNA-PK_{cs}. Although the precise targets of DNA-PK_{cs} are unknown, it has been shown to phosphorylate XRCC4 in vitro and to modulate its DNA binding activity (Leber et al, 1998; Modesti et al, 1999).

DNA-PK_{cs} is a large protein (˜3500 amino acids, M_{w} ˜465 kDa) (Smith and Jackson, 1999), the carboxyl terminus of which contains a catalytic domain that is related to that found in the phosphatidylinositol 3 (PI 3)-kinase family (Hartley et al, 1995). This similarity initially suggested that DNA-PK_{cs} might be capable of phosphorylating inositol phospholipids, but no such activity has been detected. Instead, DNA-PK_{cs} was shown to be a serine/threonine protein kinase. Other members of the PI 3-kinase related family include ATM, a protein deficient in Ataxia telangiectasia, and ATR, defects in which lead to an AT-related disorder (Keith and Schreiber, 1995; Smith and Jackson, 1999). Why these proteins should have retained the protein motifs characteristic of a phosphatidylinositol kinase remains a mystery.

WO 90/00057 relates to a method for moderating the rate of cellular mitosis in a living mammalian tissue having a pathologically elevated rate of cellular mitosis, which comprises perfusing the tissue with inositol hexaphosphate (or salt) and a source of inositol (or salt) to moderate the elevated rate of cellular mitosis. The method allegedly is useful in human and mammalian diseases wherein NK cell activity is altered, eg tumours, other cancers including leukaemia, immunosuppressed individuals, and in viral, fungal or protozoal infections.

WO 95/05380 relates to a method of modulating selectin by administering an effective amount of inositol polyanion (including inositol hexakisphosphate) which binds to the selectin. Selectin binding is apparently associated with infection with a microorganism, malignancy or other disorders including inflammation and autoimmunity.

WO 98/30902 relates to modulation of the NHEJ system via regulation (using protein and/or natural or synthetic compounds) of the interactions of XRCC4 and DNA ligase IV, and XRCC4 and DNA-PK to effect cellular DNA repair activity. It also relates to screens for individuals predisposed to conditions in which XRCC4 and/or DNA ligase IV are deficient.

WO 99/04266 relates to the interaction of p53 with, and its phosphorylation by, ATM and related protein kinases such as ATR and DNA-PK. The activity of the proteins is shown to increase in the presence of DNA. Assays for modulators of phosphorylation by the interaction between the proteins and p53 or other proteins having similar phosphorylation sites are provided. Methods of purifying ATM or ATR are also claimed

WO 00/00644 relates to a method for increasing the susceptibility of a cell to DNA-damaging agents by using an antisense oligonucleotide so as to prevent expression of a DNA dependent protein kinase subunit This invention also relates to a method of treating a tumour in a subject, comprising administering to the subject an antisense DNA-PK oligonucleotide.

Ishikawa et al (1999) Anticancer Res. 19, 3749-3752 suggests that when IP₆ is given to mice orally it reduces initiation of skin cancer development but not its promotion.

Sarkaria et al (1998) Cancer Res. 58, 4375-4382 describes the inhibition of phosphoinositide 3-kinase related kinases (such as DNA-dependent protein kinase, ATR and ATM) by the radiosensitizing agent, wortmannin.

Karanjawala et al (1999) Curr. Biol. 9, 1501-1504 shows that NHEJ is important for chromosome stability in primary fibroblasts.

Hoekstra (1997) Curr. Opinion Gen. Develop. 7, 170-175 reviews the responses to DNA damage and regulation of cell cycle check points by the ATM protein kinase family.

Hall-Jackson et al (1999) Oncogene 18, 6707-6713 shows that ATR is a caffeine-sensitive, DNA-activated protein kinase with a substrate specificity distinct from DNA-PK

Gao et al (2000) Nature 404, 897-900 describes the interplay of p53 and XRCC4 in tumourigenesis, genome stability and development.

Featherstone & Jackson (1999) Br. J. Cancer 80, 14-19 review DNA-PK and its role in repairing DNA and maintaining genomic integrity.

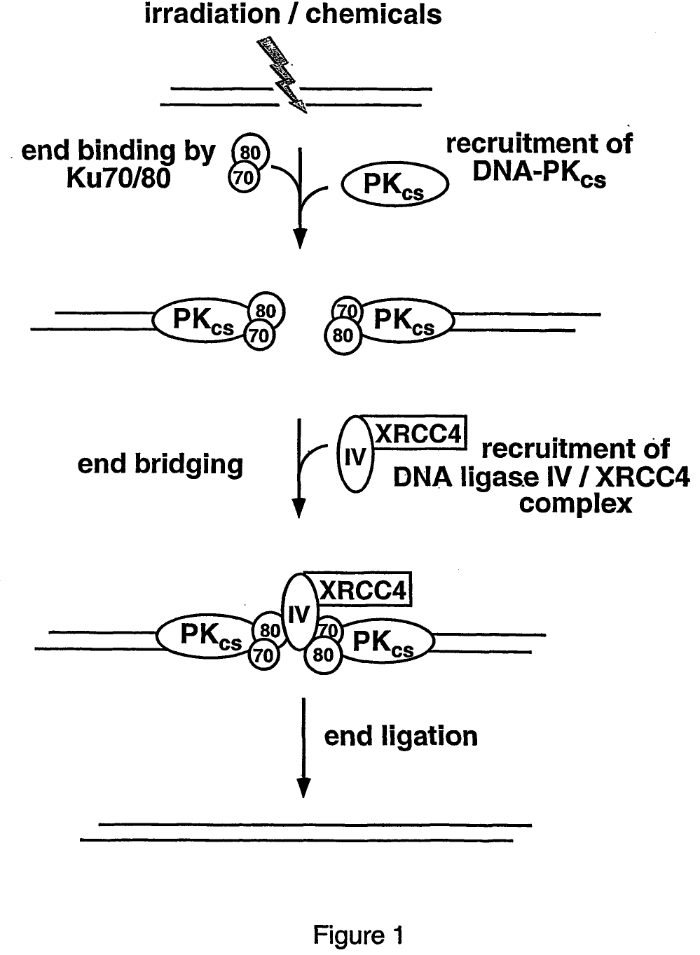

A scheme for non-homologous end-joining is shown in FIG. 1. It is thought that broken termini are recognized by the Ku heterodimer, which then recruits DNA-PK_{cs}, thereby activating its kinase activity. This large complex serves to protect the DNA ends from nuclease attack, while also facilitating the recruitment of the XRCC4/DNA ligase IV heterodimer. Although it is not at present clear how end-bridging is achieved, these reactions result in the religation of the DSB restoring the integrity of the DNA.

In an attempt to define in molecular detail the mechanism of NHEJ, an in vitro system for end-joining was recently developed (Baumann and West 1998). The reactions exhibited an apparent requirement for DNA-PK_{cs}, Ku70/80, XRCC4 and DNA ligase IV, consistent with the in vivo requirements. Preliminary fractionation and complementation assays, however, revealed that these factors were not sufficient for efficient end-joining, and that other components of the reaction remained to be identified. In the work described here, an in vitro complementation assay has been used to purify an additional component of the NHEJ reaction. The terms Ku70/Ku80 and Ku70/80 are used interchangeably to denote the heterodimer between Ku70 and Ku80 unless the context suggests otherwise.

Using a combination of phosphorus NMR, mass spectroscopy and strong anion exchange chromatography, we identify this factor as inositol hexakisphosphate (IP₆). Purified IP₆ specifically stimulates DNA-PK-dependent end joining and is bound by DNA-PK. The involvement of inositol phosphate in DNA-PK dependent NHEJ is of particular interest since the catalytic domain of DNA-PK_{cs}, is similar to that found in the phosphatidylinositol 3 (PI 3)-kinase family.

Thus, in our continued investigations of NHEJ of DNA we have surprisingly found that highly phosphorylated inositol derivatives, such as inositol hexakisphosphate (IP₆) and inositol pentakisphosphate pyrophosphate (IP₇), stimulate NHEJ. Although not wishing to be bound by any theory as to what mediates the stimulatory effect on the NHEJ of DNA, our results suggest an involvement of DNA-PK, such as a conformational change in DNA-PK that results in the stimulation of NHEJ at least in vitro. In particular, our work shows that IP₆ and IP₇ bind the Ku70/80 heterodimer which, in combination with the catalytic subunit of DNA-PK (DNA-PK_{cs}), makes up DNA-PK. IP₆ has no effect on T4 DNA ligase activity. It is possible that IP₆ binds the Ku70 subunit or the Ku80 subunit.

The present invention makes use of these observations in order to develop further methods of performing NHEJ and assays of NHEJ; screening assays for compounds which may modulate NHEJ and which may be therapeutically useful; screening assays for compound which may modulate DNA-PK and related protein kinases and which may be therapeutically useful; compositions and kits of part which may be useful in performing the assays and methods; and methods of modulating NHEJ.

The present invention also relates to methods of modulating NHEJ of DNA and therapeutic methods wherein NHEJ of DNA is enhanced or reduced. The present invention also relates to methods of measuring IP₆ or other stimulatory inositol phosphates in an individual in order to determine whether the individual may have, or be susceptible to, a defect in DNA repair or cell cycle checkpoint control.

A first aspect of the invention provides a method of stimulating non-homologous end-joining (NHEJ) of DNA the method comprising performing NHEJ of DNA in the presence of inositol hexakisphosphate (IP₆) or other stimulatory inositol phosphate. Typically, IP₆ or other stimulatory inositol phosphate is added to a NHEJ reaction in order to stimulate joining of DNA.

“Non-homologous end-joining” is the ligation of DNA termini, typically intermolecular ligation. It includes the joining of DNA ends which exhibit little or no complementarity to each other (and so, typically, each end does not hybridise to the other) and, in any event, is a term well known in the art as is evidenced by its use in many of the papers and patent applications referred to herein, all of which are incorporated herein by reference. Typically, a NHEJ reaction requires a suitable DNA substrate, and suitable components for the reaction of joining the DNA ends to proceed. Suitable DNA substrates are those that, typically, are linear DNA molecules the length of which need only be large enough to accommodate the factors which participate in NHEJ. Conveniently, each DNA fragment to be joined is, independently, at least 50 bp, preferably at least 70 bp, more preferably at least 100 bp but may be bigger. In relation to the observation of a NHEJ reaction, particularly in a screening assay, one or both of the DNA molecules (or DNA ends) to be joined are detectably labelled such as with radiolabelled phosphorus or with fluorescent labels. Although it is convenient to use two separate DNA molecules to be joined in the NHEJ, two ends of the same molecule can be joined such as the ends of a linearised plasmid. NHEJ typically takes place in a eukaryotic cell, such as a vertebrate cell including mammalian cells (although it can also occur in some circumstances in prokaryotes) but, as is described in detail in Baumann & West (1998) Proc. Natl. Acad. Sci USA 95, 14066-14070, it can also occur in cell-free extracts, such as those obtained from human cells as therein described. Intermolecular ligation in this cell-free system was found to be accurate and to depend on DNA ligase IV, XRCC4 and DNA-dependent protein kinase (DNA-PK; this is a heterotrimer made up of a catalytic subunit DNA-PKcs (encoded by the XRCC7 gene) and two further subunits which are believed to be involved in DNA binding, namely Ku70 and Ku80 subunits (which are encoded by the XRCC6 and XRCC5 genes, respectively). However, it is possible to get a low level of NHEJ with DNA ligase IV and XRCC4 in the absence of DNA-PK, but a greater extent of NHEJ is obtained when DNA ligase IV and XRCC4 are present with Ku70 and Ku80, and still further NHEJ is achieved when the catalytic subunit of DNA-PK is present. Following the inventors present work, it has now been shown that NHEJ is far better in the presence of IP₆, and even better in the presence of IP₇.

By “stimulating NHEJ” we include the meaning that the rate of NHEJ of DNA is increased by the presence of IP₆ in a NHEJ reaction mixture compared to the rate when IP₆ is not present and the reaction mixture is otherwise the same. It will be appreciated that the stimulation will reach a threshold level and that, typically, stimulation according to the method is achieved when IP₆ or other stimulatory inositol phosphate is included in a NHEJ reaction to which no IP₆ or other stimulatory inositol phosphate has been added previously. Although not being bound by any theory, it is possible that the presence of IP₆ or other stimulatory inositol phosphate is essential for NHEJ and so the presence of IP₆ or other stimulatory inositol phosphate may stimulate a NHEJ reaction from there being no joining to there being some joining of substrate DNA. It is noted that human cell-free extracts can perform NHEJ in the presence of a suitable substrate, as described in Baumann & West (1998) Proc. Natl. Acad. Sci. USA 95, 14066-14070, without the addition of IP₆; however, in this instance it is possible that the cell-free extract already contains a small amount of IP₆ or other stimulatory inositol phosphate. We have now shown that when this cell-free extract is fractionated during partial purification it loses its ability to carry out NHEJ efficiently despite containing the relevant protein components. Addition of IP₆ or IP₅ or IP₄ to the fraction (termed “PC-C” in Example 1) derived from the human cell-free extract has been shown to stimulate NHEJ of DNA. Although not being bound by any theory, it is believed that, by its chemical nature, IP₆ (and other inositol phosphates) flow through the phosphocellulose column used in the preparation of the PC-C fraction separating it from DNA-PK, XRCC4 and DNA ligase IV (see Example 1).

Thus, in a preferred embodiment the NHEJ reaction mixture contains a semi-purified cell extract which contains the necessary protein components but from which any natural stimulatory inositol phosphates have been removed. This semi-purified cell extract is then supplemented with a suitable amount of a stimulatory inositol phosphate, such as IP₆, in order to stimulate NHEJ.

By “other stimulatory inositol phosphates” we include any other inositol phosphates or derivatives of inositol phosphate (such as derivatives with one or more pyrophosphates) which have an effect on the stimulation of NHEJ which is qualitatively the same as the effect of IP₆ on the stimulation of NHEJ as defined using the reaction conditions in Example 1. Typically, the stimulatory inositol phosphate will have at least 2% of the stimulatory activity of IP₆ and preferably at least 5% or at least 10% of the stimulatory activity of IP₆ on a molar basis under the same conditions as described in Example 1. Certain stimulatory inositol phosphates may have greater stimulatory activity than IP₆; thus, the stimulatory inositol phosphate may have about the same stimulatory activity as IP₆ or it may be greater, such as 150% or 300% or 500% or even 1000% of the stimulatory activity of IP₆. Experiments described in the Examples show that IP₇ (that is to say inositol wherein five positions are occupied by phosphate residues, and one by a pyrophosphate residue) is better at stimulatory NHEJ than IP₆. IP₈ (ie inositol wherein four positions are occupied by phosphate residues and two by pyrophosphate residues) are also included as “stimulatory inositol phosphates”.

The stimulatory inositol phosphate is typically an inositol polyphosphate (ie it has several phosphate groups). The inositol phosphate may be an oligomer or polymer of inositol phosphate moieties wherein the inositol phosphate moieties are joined by a suitable linker. The oligomer or polymer may be a homo-oligomer/polymer in which case each inositol phosphate moiety is the same, or it may be a hetero-oligomer in which case at least some of the inositol phosphate moieties may be different.

It is possible to use a mixture of stimulatory inositol phosphates in order to stimulate NHEJ.

The “stimulatory inositol phosphate” may be an inositol phosphate derivative wherein one or more of the phosphate groups have been replaced by phosphonate groups.

It is possible that IP₆, when interacting with DNA-PK is modified or further phosphorylated (eg to a pyrophosphate form). Any such modification, if it leads to a stimulatory inositol phosphate as defined is included within the scope of the invention.

Derivatives of inositol phosphate include derivatives in which an inositol phosphate moiety is attached to another moiety, for example by linkage through a free hydroxyl position (if present) or through phosphate.

Typically the stimulatory inositol phosphate is a naturally-occurring inositol phosphate or derivative thereof. Preferably, the stimulatory inositol phosphate is any one of IP₆, IP₅ or IP₄.

By “IP₆” we include any stereoisomer of inositol hexakisphosphate. It is preferred that the IP₆ is myo-inositol 1,2,3,4,5,6-hexakisphosphate.

By “IP₅” we include any stereoisomer of inositol pentakisphosphate. It is preferred that the IP₅ is myo-inositol 1,3,4,5,6 pentakisphosphate.

By “IP₄” we include any stereoisomers of inositol tetrakisphosphate. It is preferred that the IP₄ is D-myo-inositol 1,3,4,5-tetrakisphosphate.

As noted, the stimulatory inositol phosphate may be IP₇ or IP₈ or an inositol phosphate with further pyrophosphate residues.

By “IP₇” we include any stereoisomer of diphosphoinositol pentakisphosphate. It is preferred that the IP₇ is myo-5-diphosphoinositol 1,2,3,4,6-pentakisphosphate (IP₇-pp5) or myo-6-diphosphoinositol 1,2,3,4,5-pentakisphosphate (IP₇-pp6).

By “IP₈” we include any stereoisomer of bis-diphosphoinositol tetrakisphosphate.

These compounds are widely available from chemical suppliers such as Sigma and Calbiochem. Shears et al (1995) J. Biol. Chem. 270, 10489-10497 discloses the synthesis of bis-diphosphoinositol tetrakisphosphate. Falck et al (1995) J. Am. Chem. Soc. 117, 12172-12175 and Reddy et al (1997) Tetrahedron Letters 38, 4951-4952 disclose the synthesis of various isomers of IP₇. Radiolabelled inositol phosphates are available from DuPont-NEN.

Other inositol phosphates, particularly those which have four, five or six phosphate groups may have substantially the same stimulatory effect in NHEJ as myo-inositol 1,2,3,4,5,6-hexakisphosphate and the skilled person will be able to determine this using the methods described in the Examples.

The methods and assays of the invention therefore include the use of such inositol phosphates with substantially the same stimulatory effect. For the avoidance of doubt, and for the purposes of the invention, myo-inositol 1,3,4,5,6 pentakisphosphate and D-myo-inositol 1,3,4,5 tetrakisphosphate are considered to have substantially the same stimulatory effect as myo-inositol 1,2,3,4,5,6 hexakisphosphate, although they are not as effective in stimulating NHEJ as the particular IP₆. IP₇ is more effective than the particular IP₆ in stimulating NHEJ.

Inositol hexasulphate (IS6) is unable to stimulate end-joining; myo-inositol 1,4,5-trisphosphate was shown to inhibit end-joining.

A second aspect of the invention provides an assay of non-homologous end-joining (NHEJ) of DNA wherein the assay comprises inositol hexakisphosphate (IP₆) or other stimulatory inositol phosphate.

The assay contains sufficient components in order to carry out NHEJ of DNA. In particular, typical assays of NHEJ are those which can be performed in vitro such as described in Baumann & West supra. NHEJ in cell-free systems is also described in Labhart (1999) Eur. J. Biochem. 265, 849-861, incorporated herein by reference. Reconstitution of NHEJ may be achieved by using recombinantly expressed protein components (such as expressed using a baculovirus system); typically, such a reconstituted system includes DNA-PK, XRCC4, DNA ligase IV, a suitable DNA substrate and a stimulatory inositol phosphate such as IP₆. The assay may also be carried out in vivo using DNA substrates which, for example, are designed to observe V(D))J joining (see Smith et al (1998) J. Mol. Biol. 281, 815-825 for an example).

It is preferred for the method of the first aspect of the invention or the assay of the second aspect of the invention (and indeed for all aspects of the invention which rely on IP₆ or other stimulatory inositol phosphate in an assay or method) that the IP₆ or other stimulatory inositol phosphate is added exogenously, although it is possible that the IP₆ stimulatory inositol phosphate is released from a source present in a cell or cell extract. Typically, the IP₆ or other stimulatory inositol phosphate is added at the start of a NHEJ reaction. Preferably, the concentration of stimulatory inositol phosphate in the reaction is between 10 nM and 50 μM; more preferably between 50 nM and 10 μM; still more preferably between 100 nM and 1 μM. These ranges are particularly preferred for IP₆.

Preferred ranges for IP₇ are between 0.1 and 1 μM.

Although the method of the first aspect of the invention or the assay of the second aspect of the invention may be carried out in vivo, it is preferred if it is carried out in vitro; in vitro methods are particularly suitable for the drug screening methods described in more detail below.

The NHEJ of DNA in the method of the first aspect of the invention and in the assay of the second aspect of the invention typically make use of a NHEJ reaction mixture which includes DNA-dependent protein kinase (and preferably all components thereof, namely DNA-PKcs, Ku70 and Ku80), XRCC4, DNA ligase IV and a suitable DNA substrate. Preferably, the NHEJ reaction contains all of these components. It may also include other components which are required for, or enhance, the reaction or make detection of the joined DNA products more readily detected. Such components include ATP and Mg²⁺ as is described in Example 1. The presence of deoxynucleotides (dNTPs) is required in NHEJ reactions in which non-complementary DNA termini were used since some processing would be expected prior to rejoining.

Amino acid and nucleic acid sequences of polypeptides useful in various aspects of the invention are available from GenBank under the following Accession Nos: human Ku70—J04611; human Ku80—M30938; human DNA ligase IV—X83441; human XRCC4—U40622; human DNA—PKcs U47077; S. cerevisiae Ku70—X70379; S. cerevisiae Ku80—Z49702; S. cerevisiae DNA ligase IV—Z74913.

It will be appreciated that this information may be used to produce the encoded proteins using standard recombinant methods as well known in the art. Thus, the methods and assays which employ a reconstituted NHEJ reaction mixture may suitably make use of recombinantly produced polypeptide components which are readily available.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral eg phage, or phagemid, as appropriate. For further details see, for example, “Molecular Cloning: a Laboratory Manual”: 2^{nd} edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al, eds. John Wiley & Sons, 1992.

Polypeptides can be purified and isolated from the host cells using techniques well known in the art.

Although it is preferred that the polypeptide components of the NHEJ reaction mixture are human (for example extracted from human cells or produced by recombinant techniques from human coding sequences, whether or not expressed in human cells), it will be appreciated that they may be from any suitable source, for example from other mammals or other vertebrates. Because of the conserved nature of the NHEJ reaction in eukaryotic cells, the components may come from lower eukaryotes such as Saccharomyces cerevisiae or Schizosaccharomyces pombe. Although it is preferred that all of the polypeptide components in a given NHEJ reaction mixture are from the same source (eg all are human), it may be possible to “mix and match” the components, for example by using a DNA ligase IV from one source and a DNA-PKcs from another source, provided that the components are able to perform NHEJ of DNA.

It will be appreciated that the polypeptide components of the NHEJ reaction mixture may have the same amino acid sequence as the polypeptide as found in nature or it may be a variant thereof which retains the necessary activity for use in a NHEJ reaction. When discussing protein kinases (below) variants are ones which retain their protein kinase activity (ie catalytic activity and/or ability to interact with another component). Variants include variants in which one or more amino acids have been inserted, deleted or replaced. A particularly useful variant is a fusion of the polypeptide with another peptide or polypeptide which facilitates purification. Such a polypeptide is the well known glutathione S-transferase. Unless the context indicates otherwise, a reference to a polypeptide includes a reference to a variant as defined. For the avoidance of doubt, the term variant included a fragment which retains a defined activity. In relation to protein kinases, in particular the PI 3-kinase related kinases described below, suitable fragments include those which retain the domain, such as a C-terminal domain, homologous to PI 3-kinase. The position of the PI 3-kinase-related domain in this family of proteins is detailed in Featherstone & Jackson (1999) Br. J. Cancer 80 (Suppl 1), 14-19.

It will be appreciated that the invention also includes aqueous compositions that contain at least one of DNA-PKcs, Ku70, Ku80, XRCC4, DNA ligase IV and a suitable DNA substrate (and preferably all of these) in addition to IP₆ or other stimulatory inositol phosphate wherein the IP₆ or other stimulatory inositol phosphate is at a concentration of at least 10 nM, preferably at least 50 nM, more preferably at least 100 nM, still more preferably at least 1 μM. At least with components derived from humans, a crude estimate of the K_{d} for IP₆ is 1 μM and so, conveniently, to saturate the system 10 μM IP₆ may be used. At this level, increases in IP₆ do not appear to result in further increase in NHEJ. The estimate of the K_{d} for IP₇ is 100 μM and saturation occurs at 1 μM.

It is particularly preferred if the polypeptide components of the aqueous composition consist only of the specific components for the NHEJ and does not include other polypeptide components, such as those derived from a cell extract, which are not required for or enhance NHEJ.

It is preferred that each polypeptide component of the aqueous composition is recombinantly produced and purified.

A third aspect of the invention provides the use of IP₆ or other stimulatory inositol phosphate for stimulating non-homologous end-joining of DNA. It is will be appreciated that before the present invention, and despite the extensive study of NHEJ, it was not realised that IP₆ or other stimulatory inositol phosphate could stimulate (or may even be essential for) NHEJ. The invention also provides the use of IP₆ or other stimulatory inositol phosphate in assays for compounds which modulate NHEJ by whatever means, and in methods which modulate NHEJ by whatever means. The assays may involve changes in NHEJ activity, changes in the recognition of substrates by the NHEJ components and/or changes in subcellular localisation of components of the NHEJ reaction such as DNA-PK, XRCC4 or DNA ligase IV.

In order to carry out the methods and assays of the invention so far described, it is convenient to make combinations of IP₆ or other stimulatory inositol phosphates with other components which are used in an NHEJ. Thus, a fourth aspect of the invention provides a kit of parts comprising IP₆ or other stimulatory inositol phosphate and one or more of a DNA-dependent protein kinase (or its constituent parts DNA-PKcs, Ku70 and Ku80), XRCC4, DNA ligase IV and a suitable DNA substrate. Typically the kit of parts comprises IP₆ or other stimulatory inositol phosphate and each of a DNA-dependent protein kinase, XRCC4, DNA ligase IV and a suitable DNA substrate. The kit of parts may also include other components that are required to carry out an NHEJ assay such as ATP, Mg²⁺ and, in some circumstances, dNTPs.

Typically, the DNA-dependent protein kinase, XRCC4 and DNA ligase IV are provided as a fraction from a cell free extract, such as the fraction as is described in Example 1. They may also be provided by expression of the individual components by recombinant means. This method is particularly preferred when the NHEJ is to be reconstituted from purified components.

In carrying out the methods and assays of the invention described so far, it is also convenient to provide IP₆ or other stimulatory inositol phosphate in combination with recombinant cells which express polypeptide components required for a NHEJ reaction. Thus, a fifth aspect of the invention provides a kit of parts comprising inositol hexakisphosphate (IP₆) or other stimulatory inositol phosphates and a host cell expressing one or more of a DNA-dependent protein kinase, XRCC4 and DNA ligase. The host cells expressing one or more of the polypeptide components of the NHEJ reaction mixture as said may be produced using standard recombinant methods and the appropriate genes/cDNAs encoding the components as is well known in the art.

In addition to showing that IP₇, IP₆, IP₅ and IP₄ stimulates NHEJ, we have also shown that, surprisingly, IP₆ and IP₇ interact with, and may modulate the activity of, protein kinases. As far as we are aware, it has not been shown previously that IP₆ and IP₇ (or indeed any other stimulatory inositol phosphates as defined) has this effect. As is described below, combinations of IP₆ and other stimulatory inositol phosphates such as IP₇ or IP₈ with a protein kinase are useful in drug screening assays and the like.

A sixth aspect of the invention provides an assay of a protein kinase wherein the assay comprises IP₆ or other stimulatory inositol phosphate. An assay of a protein kinase contains the said protein kinase and, when the catalytic activity of the protein kinase is being measured, a suitable phosphorylatable substrate (such as the target of the said protein kinase or a phosphorylatable peptide therefrom) and a suitable phosphate donor such as ATP or an analogue thereof which has a transferable γ-phosphate group. When the ability of the said protein kinase to bind to another macromolecular component (such as another protein or DNA) is being assayed, the assay contains the protein kinase and the other component. Protein kinases are known to interact with their protein substrate and with other components. These other components may be allosteric effectors and they may be macromolecular components such as other proteins or DNA.

A seventh aspect of the invention provides a kit of parts comprising a protein kinase and IP₆ or other stimulatory inositol phosphates. Typically, the protein kinase is expressed from a recombinant DNA molecule.

In the assay in the sixth aspect of the invention, and the kit of parts in the seventh aspect of the invention it is preferred that the protein kinase is substantially free of other components with which it is naturally associated.

The invention also includes aqueous compositions that contain at least one protein kinase (preferably substantially free of other components with which it is naturally associated, for example as produced by recombinant expression) and a stimulatory inositol phosphate such as IP₆ or IP₇. Preferably, the protein kinase included in an assay, kit of parts or aqueous composition is substantially pure (eg at least 90% pure).

An eighth aspect of the invention provides a kit of parts comprising IP₆ or other stimulatory inositol phosphate and a host cell expressing a protein kinase.

Protein kinases can be expressed recombinantly as is well known in the art. For example, the cloning, expression and isolation of protein kinases can be carried out using the methodology described above.

We have shown that DNA-PK binds IP₆ and IP₇ but not IP₃. More particularly, we have shown that IP₆ and IP₇ binds the Ku70/80 heterodimer portion of DNA-PK. IP₆ or IP₇ may bind to the Ku70 subunit or to the Ku80 subunit. The presence of the catalytic subunit (DNA-PK_{cs}) is not required for binding of IP₆ or IP₇ to Ku70/80. DNA-PK along with ATM, ATR and FRAP belong to a family of related protein kinases wherein the protein kinase is a protein kinase which has a domain, preferably a C-terminal domain, with similarity to the catalytic domain of phosphatidylinositol 3-kinase. DNA-PK, ATM, ATR and FRAP have an inositol head group binding domain. Other members of the family include the Saccharomyces cerevisiae gene products Tel1p, Mec1p, Tor1p and Tor2p, and the Schizosaccharomyces pombe gene product Rad3. It is preferred that the protein kinase in the sixth, seventh or eighth aspects of the invention is a member of this family, most preferably one of DNA-PK, ATR, ATM or FRAP.

ATM is the protein encoded by the gene mutated in human ataxia-telangiecstasia (or equivalent genes in other species).

Sequence analysis reveals that the human ATM gene encodes a ˜350 kDa polypeptide (Savitsky et al (1995) Science 268, 1749-175; Savitsky et al (1995) Hum. Mol. Genet. 4, 2025-2032).

As noted above, included in the family are Saccharomyces cerevisiae Tor1p and Tor2p and their mammalian homologue FRAP, which control progression into S-phase and, at least in part, function by regulating translation (Brown and Schreiber (1996) Cell 86, 517-520). Also in this family is the DNA dependent protein kinase (DNA-PK) catalytic subunit (DNA-PKcs), defects in which lead to sensitivity to IR and an inability to perform site-specific V(D)J recombination (reviewed in Jackson and Jeggo (1995) Trends Biochem. Sci 20, 412-415; Jackson (1996) Curr. Opinion Genet. Dev. 6, 19-25. Other members of the ATM sub-group of the PI 3-kinase family that have been identified include S. cerevisiae Tel1p and Mec1p, together with the Mec1p homologues of Schizosaccharomyces pombe (rad3), Drosophila melanogaster (mei-41) and humans FRP1/ATR; (Keith and Schreiber (1995) Science 270, 50-51; Zakian (1995) Cell 82, 685-687; Jackson (1996) Curr. Opinion Genet. Dev. 6, 19-25). As with ATM, defects in these proteins lead to genomic instability, hypersensitivity towards DNA damaging agents and defects in DNA damage-induced cell cycle checkpoint controls.

The Gen Bank Accession Nos for cDNAs encoding various of these protein kinases is as follows:

FRAP human cDNA NM_{—}004958

ATR human cDNA NM_{—}001184 and U76308

ATM human cDNA NM_{—}000051

TEL1 S. cerevisiae Z35849 Y13134

MEC1 S. cerevisiae U31109

MEI-41 Drosophila U34925

RAD3 S. pombe U76307

WO 99/04266, incorporated herein by reference, describes methods of purifying ATM and ATR.

It is believed that members of the family (which may be termed PI3 kinase related kinases) share, in their C-terminal domain at least 25% amino acid sequence identity with the C-terminal domains of DNA-PKcs, more preferably at least 30% or 35% or 40% or 50% or 70% or 90% sequence identity. When compared with the PI3-kinase domain of ATM, DNA-PK shows 28% homology, and RAD3 shows 39% (see Hunter (1995) Cell 83, 1-4). The carboxy termini of the relevant proteins are compared in Keith & Schreiber (1995) Science 270, 50-51.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., 1994). The parameters used may be as follows:

Fast pairwise alignment parameters: K-tuple (word) size; 1, window size; 5, gap penalty; 3, number of top diagonals; 5. Scoring method: x percent. Multiple alignment parameters: gap open penalty; 10, gap extension penalty; 0.05.

Scoring matrix: BLOSUM.

NHEJ is involved in many important biological processes, and modulation of NHEJ has been proposed in relation to a number of medical practices. Compounds that modulate these processes may be useful as potential drugs or for further studies on NHEJ. Thus, these compounds may be useful for any of a variety of purposes.

One is anti-tumour or anti-cancer therapy, particularly augmentation of radiotherapy or chemotherapy. Ionising radiation and radiomimetic drugs are commonly used to treat cancer by inflicting DNA damage. Cells deficient in DNA repair are hypersensitive to ionising radiation and radiomimetics. Chemotherapy includes the use of topoisomerase II poisons and other compounds involved in DNA control. Another is the potentiation of gene targeting and gene therapy. Modulation of NHEJ may be used to increase efficiencies of gene targeting, of interest and ultimate use in gene therapy.

The frequency of spontaneous allelic recombination in higher eukaryotic cells is extremely low, and poses a major limitation to its therapeutic potential. Gene therapy by in vivo or ex vivo gene targeting may therefore remain impractical unless dramatic improvements in targeting efficiency can be achieved. For many gene therapy applications, it is desirable to be able to target the integration of the transgene in a specific locus of the host genome. Enhancing the frequency of homologous recombination in the target cells, through inhibition of NHEJ, may prepare the cells for targeted gene integration by homologous recombination.

A further, related, purpose is in anti-retroviral therapy, since DNA repair pathways such as involving the components XRCC4 and DNA ligase IV are involved in effecting retroviral and retrotransposon integration into the genome of a host cell. Retroviruses are of considerable risk to the health of humans and animals, causing, inter alia, AIDS, various cancers and human adult T-cell leukaemia/lymphoma. Integration of retroviral DNA into the genome is essential for efficient viral propagation and may be targeted by inhibition of DNA repair pathway components.

Additionally, modulators of NHEJ may be used in modulation of immune system function, since such factors are required for generation of mature immunoglobulin and T-cell receptor genes by site-specific V(D)J recombination.

Thus, further methods for identifying such compounds are useful for the pharmaceutical industry.

A ninth aspect of the invention provides a method of identifying a compound which modulates or mimics the effect of inositol hexalisphosphate (IP₆) or other stimulatory inositol phosphate in stimulating non-homologous end-joining (NHEJ) of DNA the method comprising performing NHEJ of DNA in the presence of inositol hexakisphosphate (IP₆) or other stimulatory inositol phosphate and determining the effect of a test compound on the NHEJ of DNA.

Typically, the method is carried out in vitro, for example, using a suitable fraction from a cell-free extracts as described in Example 1, or by using reconstituted systems for NHEJ as described above. The method may also be carried out in vivo as described above.

Typically the NHEJ of DNA is carried out in a NHEJ reaction mixture which includes DNA-PK, XRCC4, DNA ligase IV and a suitable DNA substrate. Preferably, DNA-PK, XRCC4 and DNA ligase IV are the only polypeptide components of the reaction mixture. Preferably, the DNA-PK, XRCC4 and DNA ligase IV are each substantially pure (eg at least 90% pure) before combination.

In a preferred embodiment, NHEJ is measured using the methods described in Example 1. In particular, the DNA substrates which are joined by the NHEJ reaction are preferably radioactively labelled and the joining is measured by size-separating the DNA, and for example by agarose gel electrophoresis, and the DNA detected by phosphorimaging. High throughput screening assays may be developed, and an example is given in Example 2.

Typically, the NHEJ is based on the ligation of two linear DNA molecules (although ligation/recircularisation of a linearised plasmid may be measured). High throughput screens may be based on the detection of the retention of a labelled (eg radiolabelled or fluorescently labelled) DNA molecule on a solid support (eg microtitre well) following ligation to a second DNA molecule immobilised on the solid support. PCR amplification across the ligation junction (hence producing a product only following ligation) may also be used.

Typically, a series of reactions are carried out which assess the effect of the test compound in order to confirm (or deny) that it is a compound which specifically modulates or mimics the effect of the stimulatory inositol phosphate on the NHEJ of DNA rather than a compound which has a nonspecific effect. Thus, for example, the test compound may be added to the NHEJ reaction either before or after the addition of IP₆ or other stimulatory inositol phosphate such as IP₇ to determine whether the order of addition has an effect on the NHEJ reaction. Alternatively, or additionally, comparisons may be made between a reaction which contains the test compound and contains IP₆ or other stimulatory inositol phosphate and reactions which do not contain IP₆ or other stimulatory inositol phosphate or do not contain the test compound.

Compounds which specifically enhance the effect of the stimulatory inositol phosphate in the NHEJ typically lead to increased NHEJ activity, whereas compounds which specifically reduce the effect of the stimulatory inositol phosphate in the NHEJ typically lead to decreased NHEJ activity. Compounds which mimic the stimulatory inositol phosphate in a structural sense may increase the NHEJ activity if the mimic is a functional analogue but may decrease NHEJ activity if the mimic is not a functional analogue.

The methods of the ninth aspect of the invention are suitable for identifying compounds which modulate or mimic the effect of IP₆ or other stimulatory inositol phosphate on the catalytic (ie DNA joining activity) of the NHEJ reaction. The invention also includes the identification of compounds that modulate the interactions between components of a NHEJ reaction whether or not such modulation leads directly to a change in DNA joining activity. Thus, a tenth aspect of the invention provides a method of identifying a compound which modulates or mimics the effect of inositol hexakisphosphate (IP₆) or other stimulatory inositol phosphate in stimulating non-homologous end-joining (NHEJ) of DNA the method comprising determining, in the presence of inositol hexalisphosphate (IP₆) or other stimulatory inositol phosphate, the effect of a test compound on the interactions between the components NHEJ reaction.

In a preferred embodiment of the invention the components of the NHEJ reaction between which an interaction is measured is any one or more of a DNA-dependent protein kinase (or its components ie DNA-PK_{cs}, Ku70 and Ku80), XRCC4, DNA ligase IV and a suitable DNA substrate. Other interactions include interaction of any one of XRCC4, DNA ligase IV and the DNA substrate with any one of Mre11, NBS and Rad50 which themselves form a complex (Mre11/NBS/Rad50). The Mre11/NBS/Rad50 complex is believed to net act upstream of DNA-PK in the processing of DNA ends. Further details of the Mre11/NBS/Rad50 complex are found in Labhart (1999) Eur. J. Biochem. 265, 849-861.

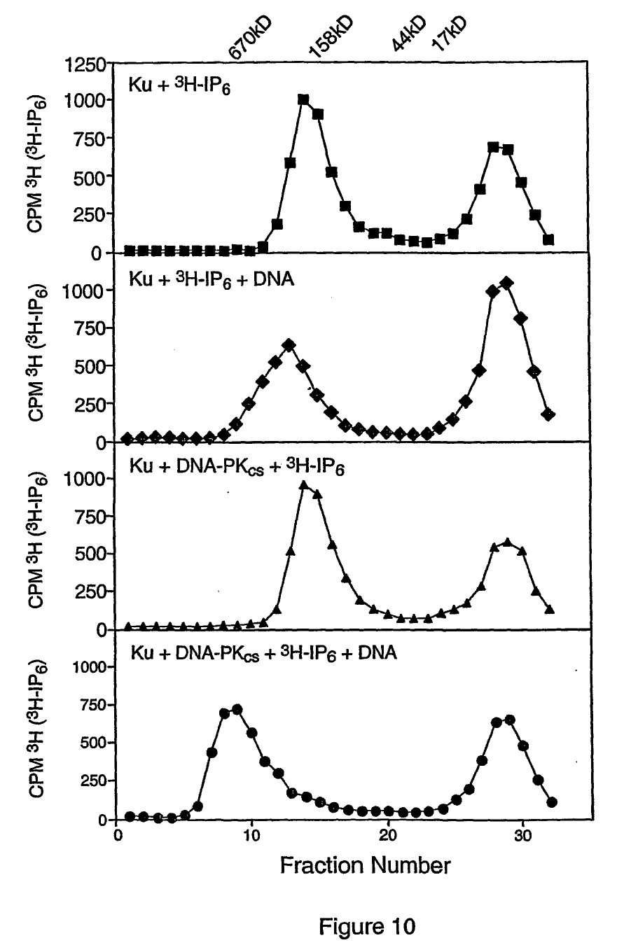

Since we have determined in Example 4 that IP₆ and other stimulatory inositol phosphates such as IP₇ bind to the Ku70/80 heterodimer of DNA-PK, it is particularly preferred that this heterodimer is present and that interactions between it and other NHEJ components are measured. The interaction between Ku 70/80 and DNA-PK_{cs} may be measured, as may the interaction between Ku 70/80 and DNA. Some of these interactions also require the presence of DNA. For example, Ku70/80-IP₆ and DNA-PK_{cs} may require DNA to form a supercomplex (see FIG. 10). The interaction between the Ku70 and Ku80 subunits in the heterodimer may also be measured.

Details of methods of measuring interactions between components, such as protein-protein interactions, protein-DNA interactions and protein-small molecule interactions are described below following the discussion of the aspect of the invention relating to other protein kinases. However, in relation to the present aspect of the invention, conveniently a series of measurements are made which assess the effect of the test compound in order to confirm (or deny) that it is a compound which specifically modulates or mimics the effect of IP₆ or other stimulatory inositol phosphate on the interaction between components of a NHEJ reaction rather than a compound which has a non-specific effect. Thus, for example, the test compound may be added to the sample containing components of the NHEJ reaction whose interaction is to be measured either before or after the addition of IP₆ or other stimulatory inositol phosphate to determine whether the order of addition has an effect on the interaction between components. Alternatively, or additionally, comparisons may be made between the interactions in a sample containing components of the NHEJ reaction whose interaction is to be measured which sample contains the test compound and IP₆ or other stimulatory inositol phosphate with equivalent samples (in terms of the NHEJ components) which do not contain IP₆ or other stimulatory inositol phosphate or do not contain the test compound.

Compounds which modulate or mimic the effect of the stimulatory inositol phosphate can be selected by their activity to modulate or mimic the interactions of the components when in the presence of the stimulatory inositol phosphate.

Preferred embodiments of this aspect of the invention measure the interaction (in the presence of IP₆ or other stimulatory inositol phosphate) between DNA-PK and its DNA effector; DNA-PK and a cosubstrate (such as ATP); DNA-PK and XRCC4; DNA-PK and DNA ligase IV; XRCC4 and DNA ligase IV; and DNA ligase IV and its DNA substrate and/or a cosubstrate such as ATP. Further preferred embodiments of this aspect of the invention measure the interaction (in the presence of IP₆ or other stimulatory inositol phosphate) of Ku70/80 with DNA-PK_{cs}, and the interaction between Ku70 and Ku80 (whether or not in the presence of DNA-PK_{cs}). The interaction between Ku70/80 and DNA-PK_{cs} may require DNA.

Interactions between any one of these components and any one of the components of the Mre11/NBS/Rad50 complex may also be measured.

The measurement of interactions between certain of these components is described in WO 98/30902 and these measurement techniques are incorporated herein by reference. It should be noted that, as discussed above, there is no realisation in WO 98/30902 of the importance of IP₆ or other stimulatory inositol phosphate in NHEJ of DNA or in the interaction between components required for NHEJ. In this aspect of the invention, it is particularly preferred if the method is used to identify compounds which modulate or mimic the effect of IP₆ or other stimulatory inositol phosphate in stimulating NHEJ by altering the interaction of DNA-PK with another component (such as its effector DNA or DNA ligase IV or XRCC4 or its cognate DNA or a substrate or cosubstrate) or by altering the interactions between the components of DNA-PK (ie DNA-PKcs and Ku70 and/or Ku80).

An eleventh aspect of the invention provides a method of identifying a compound which modulates or mimics the effect of inositol hexakisphosphate (IP₆) or other stimulatory inositol phosphate on a protein kinase the method comprising determining, in the presence of IP₆ or other stimulatory inositol phosphate, the effect of a test compound on the catalytic activity of the protein kinase or on the ability of the protein kinase to interact with another component.

Protein kinases phosphorylate, generally in a specific manner, proteins in hydroxyl-containing amino acid residues (serine, threonine or tyrosine), by transferring the γ-phosphate group from ATP to the protein. The substrate for the protein kinase is typically the cognate protein but, conveniently, it may be a synthetic peptide derived from the protein and which contains the phosphorylatable amino acid residue. Protein kinases are also able to autophosphorylate.

Typically, the protein kinase is a protein kinase which has a domain, preferably a C-terminal domain, with similarity to the catalytic domain of phosphoinositide 3-kinase. Conveniently, the protein kinase is a protein kinase involved in the maintenance of genome stability, such as those which are involved in a DNA repair response. Preferably, the protein kinase is any one of a DNA-dependent protein kinase, ATR, ATM, FRAP, or the Saccharomyces cerevisiae gene products Tel1p, Mec1p, Tor1p or Tor2p, or the Schizosaccharomyces pombe gene product Rad3. It is particularly preferred if the protein kinase is any one of DNA-PK, ATR, ATM or FRAP, and most preferred if the protein kinase is DNA-PK.

DNA-PK is known to phosphorylate XRCC4 which is, therefore, a suitable substrate. Peptide portions of XRCC4 may also be suitable as substrates.

Each of DNA-PK, ATR, ATM and FRAP can phosphorylate p53 in vitro. This phosphorylation has been mapped to Ser15 in p53 for ATM, ATR and DNA-PK so an N-terminal peptide of p53 may be used as a substrate (Hall-Jackson et al (1999) and references cited therein). Yarosh et al (2000) J. Invert. Dermatol. 114, 1005-1010 shows that FRAP is a DNA-dependent protein kinase which is associated with UV-induced damage. FRAP also phosphorylates PHAS-1 (Brunn et al (1997) Science 277, 99-101).

Catalytic activity of a protein kinase, such as DNA-PK, ATR, ATM or FRAP, can readily be determined using methods well known in the art, such as by measuring the incorporation of a radiolabelled phosphate group in the substrate following transfer from ATP. Typically, a series of reactions are carried out which assess the effect of the test compound in order to confirm (or deny) that it is a compound which modulates or mimics the effect of IP₆ or other stimulatory inositol phosphate on a protein kinase rather than a compound which has a non-specific effect. Thus, for examples the test compound may be added to the protein kinase reaction either before or after the addition of IP₆ or other stimulatory inositol phosphate to determine whether the order of addition has an effect on the catalytic activity. Alternatively, or additionally, comparisons may be made between a reaction which contains the test compound and IP₆ or other stimulatory inositol phosphate and reactions which do not contain IP₆ or other stimulatory inositol phosphate or do not contain the test compound.

The catalytic activity of the protein kinase can be assessed by the transfer of the γ-phosphate from ATP to a substrate (which may include itself ie autophosphorylation). Typically, the γ-phosphate is radiolabelled and so phosphorylation of the substrate can be detected by detecting the radioactivity (eg by scintillation counting, autoradiography or phosphorimaging). Alternatively, it can be detected by separating the phosphorylated substrate from non-phosphorylated substrate, or by using phosphoprotein specific antibodies, for example by fluorescence.

When the effect of a test compound on the interaction between a protein kinase and another component is determined, it is particularly preferred if the protein kinase is the catalytic subunit of DNA-PK (ie DNA-PKcs) and the other component is any one of Ku70, Ku80, DNA ligase IV, XRCC4 or a suitable DNA effector thereof. Preferably, when the protein kinase is DNA-PK, the other component is XRCC4.

The protein kinases ATM and ATR, like DNA-PK, are activated by DNA. FRAP can be stimulated by a DNA damage response. Thus, in a further preferred embodiment, the protein kinase is DNA-PK, ATM, ATR or FRAP and the interaction with its effector DNA is determined.

Typically, a series of measurements are made which assess the effect of the test compound in order to confirm (or deny) that it is a compound which specifically modulates or mimics the effect of IP₆ or other stimulatory inositol phosphate on the protein kinase rather than a compound that has a non-specific effect. Thus, for example, the test compound may be added to a sample containing the protein kinase and a compound with which it interacts (such as its substrate but in the absence of ATP or such as an effector DNA as is the case with DNA-PK or ATR or ATM or FRAP) before or after the addition of IP₆ or other stimulatory inositol phosphate to determine whether the order of addition has an effect on the interaction between components. Alternatively, or additionally, comparisons may be made between the interactions in a sample containing the protein kinase and a component with which it interacts which sample contains the test compound and IP₆ or other stimulatory inositol phosphate with equivalent samples (in terms of protein kinase and interacting component) which do not contain IP₆ or other stimulatory inositol phosphate or do not contain the test compound.

A component with which a protein kinase interacts includes ATP or an analogue thereof, such as a non-hydrolysable analogue as is well known in the art.

A further aspect of the invention provides a method of identifying a compound which modulates the binding of IP₆ or other stimulatory inositol phosphate to a protein kinase, the method comprising determining whether a test compound reduces or increases the binding of IP₆ or other stimulatory inositol phosphate to the said protein kinase or a subunit thereof.

It is particularly preferred that the subunit of the protein kinase is the Ku70/Ku80 heterodimer of DNA-PK.

The interaction (association) of the stimulatory inositol phosphate (such as IP₆) with, and dissociation from the protein kinase or subunit thereof may be measured using methods well known in the art. Typically, the protein kinase is immobilised and the binding of detectably-labelled stimulatory inositol phosphate is measured. Typically, the stimulatory inositol phosphate, such as IP₆, is radiolabelled or fluorescently labelled. Scintillation proximity assays, as described below, are particularly useful in binding assays.

We have shown that IP₆ and IP₇ binds to DNA-PK. More particularly, IP₆ and IP₇ have been shown to bind to the Ku70/80 heterodimer which forms part of DNA-PK. Thus, it is particularly preferred that the protein kinase is a protein kinase which has a domain, preferably a C-terminal domain, with similarity to the catalytic domain of phosphoinositide 3-kinase. The protein kinase is preferably any one of the members of this family of protein kinases as discussed above. It is particularly preferred if it is DNA-PK. As noted DNA-PK is made up of three subunits. The binding assay may use any one of DNA-PK_{cs}, Ku70 or Ku80 or, as noted above, any functional variants (eg fragments) thereof which retain the binding site for the stimulatory inositol phosphate such as IP₆. Preferably, the binding assay uses the Ku70/Ku80 heterodimer.

A still further aspect of the invention provides a method of identifying a compound which modulates the binding of IP₆ or other stimulatory inositol phosphate to the Ku70/Ku80 heterodimer of DNA-PK or Ku70 subunit thereof or Ku80 subunit thereof, the method comprising determining whether a test compound reduces or increases the binding of IP₆ or other stimulatory inositol phosphate to the said Ku70/Ku80 heterodimer or the Ku70 subunit thereof or the Ku80 subunit thereof.

Preferably, the Ku70/Ku80 heterodimer is used.

In one embodiment the inositol phosphate (for example, IP₅ or IP₆ or IP₇) is immobilised onto a solid substrate (such as the floor and/or wall of a microtitre plate) and the Ku 70/80 heterodimer is bound thereto. A competition binding assay is carried out with test compounds. The Ku 70/80 used may be suitably detectably labelled so that the presence or absence (or depletion) of Ku 70/80 bound to the inositol phosphate can readily be detected.

Additionally or alternatively, a detectably labelled antibody which selectively binds to Ku 70/80 (or to either component thereof) may be used to detect the presence or absence (or depletion) of Ku 70/80 bound to the inositol phosphate. Suitable antibodies are commercially available from Neo Markers, LabVission Corporation, 47790 Westinghouse Drive, Fremont, Calif. 94539, USA.

It will be appreciated that in this aspect of the invention the Ku70/80 heterodimer or Ku70 subunit thereof or Ku80 subunit thereof do not require the presence of DNA-PS. The Ku protein (independent of DNA-PK_{cs}) is involved in telomere biology and, in particular, Peterson et al (2001) Nature Genet. 27, 64-67 indicate that the function of a stem-loop in telomerase RNA is linked to the DNA repair protein Ku. Thus, compounds which modulate the binding of IP₆ or other stimulatory inositol phosphate to Ku70/80 heterodimer or Ku70 subunit thereof or Ku80 subunit thereof may modulate teleomeres or telomerase function.

Preferably, the compound is one which modulates the binding of IP₆ or other stimulatory inositol phosphate to the Ku70/80 heterodimer.

A still further aspect of the invention provides a method of identifying a compound which modulates the binding of IP₆ or other stimulatory inositol phosphate to XRCC4 or DNA ligase IV, the method comprising determining whether a test compound reduces or increases the binding of IP₆ or other stimulatory inositol phosphate to the said XRCC4 or DNA ligase IV.

Binding of the stimulatory inositol phosphate to XRCC4 or DNA ligase IV can be done using analogous methods as for binding to protein kinases.

The amount of test compound or substance which may be added to a screening method or assay of the invention will normally be determined by trial and error depending on the type of compound or method used. Typically, the test compound may be used at a concentration of around 0.01 nm to 100 μM. Compounds which may be used as test compounds may be natural or synthetic compounds. Extracts of plants which may contain several characterised or uncharacterised compounds may also be used and are considered “test compounds”.

It will be appreciated that screening assays which are capable of high throughput operation will be particularly preferred. Examples may include cell based assays and protein-protein binding assays. An SPA-based (Scintillation Proximity Assay; Amersham International) system may be used. For example, an assay for identifying a compound capable of modulating the activity of a protein kinase may be performed as follows. Beads comprising scintillant and a polypeptide that may be phosphorylated may be prepared. The beads may be mixed with a sample comprising the protein kinase and ³²P-ATP or ³³P-ATP and with the test compound. Conveniently this is done in a 96-well format. The plate is then counted using a suitable scintillation counter, using known parameters for ³²P or ³³P SPA assays. Only ³²P or ³³P that is in proximity to the scintillant, i.e. only that bound to the polypeptide, is detected. Variants of such an assay, for example in which the polypeptide is immobilised on the scintillant beads via binding to an antibody, may also be used.

Methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

Alternative methods of detecting binding of a polypeptide to macromolecules, for example DNA, RNA, proteins and phospholipids, include a surface plasmon resonance assay, for example as described in Plant et al (1995) Analyt Biochem 226(2), 342-348. Methods may make use of a polypeptide that is labelled, for example with a radioactive or fluorescent label.

The yeast two-hybrid system may be used to detect interactions between polypeptides.

An example of the use of the yeast two-hybrid system is the use of two compounds, such as two components of the NHEJ reaction mixture, and, which interact to form a complex involved in NHEJ, to facilitate the identification of compounds that modulate NHEJ. These compounds are detected by adapting yeast two-hybrid expression systems known in the art for use as described herein. These systems which allow detection of protein interactions via a transcriptional activation assay, are generally described by Gyuris et al, Cell 75:791-803 (1993) and Fields & Song, Nature 340:245-246 (1989), and are commercially available from Clontech (Palo Alto, Calif.). The yeast two-hybrid assay is carried out in the presence of IP₆ in order to identify compounds which modulate or mimic the effect of IP₆ on NHEJ.

In this approach, a region of, for example, DNA-PKcs, which interacts with, for example, Ku70, is fused to the GALA-DNA-binding domain by subcloning a DNA fragment encoding this into the expression vector, pGBT9, provided in the MATCHMAKER Two-Hybrid System kit commercially available from Clontech (catalogue number K1605-1). A fusion of the GAL4 activation domain with the region of Ku70 (which interacts with the region of DNA-PKcs) is generated by subcloning the Ku70 domain-encoding DNA fragment into the expression vector, PGAD424, also provided in the Clontech kit. Analagous expression vectors may also be used. Yeast transformations and colony lift filter assays are carried out according to the methods of MATCHMAKER Two-Hybrid System and various methods known in the art. Prior to the colony filter assay, transformed yeast may be treated with candidate compounds and, as appropriate, with IP₆ or other stimulatory inositol phosphate being screened for the ability to modulate NHEJ. The interaction results obtained using the candidate compound in combination with the yeast system may then be compared to those results observed with the yeast system not treated with the candidate compound (or not treated with IP₆), all other factors (eg cell type and culture conditions) being equal. A compound capable of modulating NHEJ is able to alter the interaction between DNA-PKcs and Ku70.

In another embodiment of this approach, a compound capable of decreasing NHEJ by disrupting the binding of DNA-PKcs to the Ku70 may be isolated using the modified yeast two-hybrid system described above, in which the reporter gene encodes a protein, such as ricin, that is toxic to yeast. Yeast cells containing such a ricin reporter die unless the binding of DNA-PKcs to Ku70 is disrupted. Yeast cells treated with a compound that disrupts the DNA-PKcs/Ku70 interaction form viable colonies, and from this result it may be inferred that the compound is capable of decreasing, and possibly inhibiting, NHEJ. Again, the assay is carried out, as appropriate, with IP₆ or other stimulatory inositol phosphate to determine that the compound is one which modulates or mimics the effect of IP₆ or other stimulatory inositol phosphate on NHEJ.

It will be appreciated that analogous reactions can be carried out with respect to protein kinase interactions with other components.

It will be appreciated from the foregoing that the present invention relates to screening methods for drugs or lead compounds.

It will be appreciated that in the methods described herein, which may be drug screening methods, a term well known to those skilled in the art, the compound selected following the screen may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The drug-like compound may have increased stability and lower toxicity than, for example, IP₆. It may also have an improved stimulatory profile compared to IP₆.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

While not being bound by any theory as to the chemical nature of compounds which may be found in the screening methods of the invention, it is preferred if the test compounds are inositol derivatives, more preferably inositol phosphates. In particular, the test compounds may be phosphoinositides or analogues of IP₆. The test compounds may be any test compounds, provided that they can be introduced into a suitable assay. Libraries of test compounds may be designed by reference to the general structure and charge distribution of IP₆, and libraries of compounds may be synthesised using combinatorial chemistry techniques as are well known in the art. It will be appreciated that the test compound is not the stimulatory inositol phosphate present in the assay. It may be possible to select test compounds for screening in the assays and methods of the invention, for example by using computer aided design to select compounds which eg have a similar spatial structure and/or charge distribution to IP₆.

The compounds identified in the screening assays of the invention may themselves be useful in medical treatment or may be useful in developing agents for medical treatment In particular, the compounds identified by the screening methods of the invention maybe useful in developing agents for the treatment of conditions where there is abnormal or inappropriate non-homologous end-joining of DNA. It is envisaged that the compounds may be useful in the development of agents for treating cancer, augmenting cancer radiotherapy and/or chemotherapy regimes, improving gene therapy regimes, enhancing homologous recombination, treating retroviral infections, and modulating the immune system.

Compounds which, following the screening method of the ninth and tenth aspects of the invention, are ones which mimic or modulate the effect of IP₆ or other stimulatory inositol phosphate in a NHEJ reaction are selected for further study. Compounds which modulate the binding of the stimulatory inositol phosphate (such as IP₆) to DNA-PK (or a subunit thereof) or XRCC4 or DNA ligase IV may also be selected for further study. Conveniently, these compounds are then tested further in another screen which is designed for the selection of compounds which are suitable for treating cancer, augmenting cancer radiotherapy and/or chemotherapy regimes, improving gene therapy regimes, enhancing homologous recombination, treating retroviral infections, or modulating the immune system. Typically, the screens are ones which involve cell-based assays which look at end-points relevant to the condition in question. The screens may also involve animal models of the relevant condition. Cell-based screens and animal models are available for at least some of cancer, augmentation of cancer radiotherapy and/or chemotherapy, gene therapy, homologous recombination, retroviral infections and immune system modulation.

Compounds which, following the screening method of the eleventh aspect of the invention, are ones which mimic or modulate the effect of IP₆ or other stimulatory inositol phosphate on protein kinases. Conveniently, these compounds are then tested in another screen which is designed for the selection of agents which modulate protein kinase or its interactions in a preferred embodiment of the invention, the protein kinase is any one of DNA-PK, ATM, ATR or FRAP and the method of the invention is used to identify compounds which may be useful in developing agents which modulate cell cycle checkpoint control. Conveniently, further screens for compounds selected in the method of the eleventh aspect of the invention include screens specifically designed to identify agents which modulate cell cycle checkpoint control.

A twelfth aspect of the invention provides a compound identifiable by the screening methods of the invention. A thirteenth aspect of the invention provides a compound identified by the screening methods of the invention. Such compounds are useful in medicine, particularly in the conditions mentioned above. The compounds may be packaged and presented for use in medicine, or may be used in the manufacture of a medicament for treating conditions in which the patient may benefit from modulation of non-homologous end-joining of DNA or from modulation of protein kinase function. For example, compounds obtained in the screens may disrupt DNA-PK-IP₆ interaction and thereby sensitise cells to therapy (eg cancer chemo- or radiotherapy) by disrupting NHEJ.

A fourteenth aspect of the invention provides a method of reducing non-homologous end-joining (NHEJ) of DNA the method comprising reducing the amount of or inhibiting the stimulatory effect of, inositol hexakisphosphate (IP₆) or other stimulatory inositol phosphate in a NHEJ reaction.

As noted above, myo-inositol trisphosphate (IP₃) is inhibitory in a NHEJ and may be used to reduce NHEJ.

A fifteenth aspect of the invention provides a method of enhancing non-homologous end-joining (NHEJ) of DNA the method comprising increasing the amount of, or enhancing or mimicking the stimulatory effect of, inositol hexakisphosphate (IP₆) or other stimulatory inositol phosphate in a NHEJ reaction.

The methods may be used in vitro, but it is particularly preferred if they are used in vivo, for example in a cell where it is desirable to reduce or enhance NHEJ of DNA. It is preferred if the cell where reduction or enhancement of NHEJ of DNA takes place according to the method of the invention is a cell in a human or animal in need of reduction or enhancement of NHEJ of DNA. As noted above, conditions where reduction of NHEJ of DNA in an animal or human may be desired include cancer, augmentation of cancer radiotherapy and/or chemotherapy, gene therapy, homologous recombination, retroviral infections and immune system modulation. Thus, the invention includes a method of treating these conditions or carrying out these procedures, the method comprising administering to the patient an effective amount of a compound which reduces the amount of, or enhances the stimulatory effect of, IP₆ or other stimulatory inositol phosphate in NHEJ reaction. The invention also includes the use of a compound which reduces the amount of, or enhances the stimulatory effect of, IP₆ or other stimulatory inositol phosphate in NHEJ reaction in the manufacture of a medicament for treating a condition where reduction of NHEJ of DNA is desired.

It is desirable to reduce NHEJ in order to enhance double-stranded break repair by homologous recombination, leading to more efficient gene targeting.

Histamine has been shown to affect IP₆ levels in cells (Sakuma et al (1988) Pharmacol. Exp. Ther. 247,466-472).

Anti-inositol phosphate antibodies may be used to reduce the amount of, or inhibit the stimulatory effect, of the stimulatory inositol phosphate, such as IP₆. Anti-inositol phosphate antibodies may be made using methods well known in the art using the inositol phosphate as an immunogen (or hapten) or by using it to screen synthetic antibody-display libraries (eg phage display libraries). By “antibody” we include antibody-like molecules such as antigen binding fragments of antibodies or synthetic antibodies. Thus, the term antibody includes Fab, Fv, ScFv, dAb and the like as are well known in the art. Antibodies to IP₃ are described in Shieh & Chen (1995) Biochem. J. 311, 1009-1014 and Goa et al (1994) Biorg. Medicinal Chem. 2, 7-13 and antibodies to IP₆ may be made by analogous methods.

The screening methods of the invention may be used to identify high affinity IP₆ analogues which may specifically compete with IP₆ for the DNA-PK binding site.

IP₆ levels in the cell may be up- or downregulated by targeting the synthetic enzyme (eg IPK1 in Saccharomyces cerevisiae) or enzymes involved in IP₆ turnover (eg phosphatases). Antisense compounds, which are well known in the art and may be designed by reference to a particular nucleotide sequence (eg to the mRNA or gene encoding the IP₆ synthetic enzyme or to an IP₆ phosphatase), may be used.

Enhancement of NHEJ may be desirable in patients who are cancer prone or who are immuno-compromised. It may also be desirable in A-T patients. Thus, the invention also includes a method of treating these conditions, the method comprising administering to the patient an effective amount of a compound which increases the amount of, or enhances or mimics the stimulatory effect of, IP₆ or other stimulatory inositol phosphate in a NHEJ reaction. The invention also includes the use of a compound which increases the amount of, or enhances or mimics the stimulatory effect of, IP₆ or other stimulatory inositol phosphate in a NHEJ reaction in the manufacture of a medicament for treating a condition where enhancement of NHEJ of DNA is desired.

As noted above, histamine has been shown to alter IP₆ levels in a cell, and antibodies may be used to reduce the amount of, or inhibit the stimulatory effect of the stimulatory inositol phosphate.

A sixteenth aspect of the invention provides a method of modulating the activity or interaction of a protein kinase the method comprising changing the amount of inositol hexakisphosphate (IP₆) or other stimulatory inositol phosphate present with the protein kinase, or inhibiting or enhancing the effect of IP₆ or other stimulatory inositol phosphate on the protein kinase.

Typically, the protein kinase is a protein kinase which has a domain, preferably a C-terminal domain, with similarity to the catalytic domain of phosphatidylinositol 3-kinase.

The method may be used in vitro, but it is particularly preferred if it is used in vivo, for example in a cell where it is desirable to modulate the activity or interaction of the protein kinase. Typically, the modulation of the protein kinase takes place in a cell in a human or animal body, in need of such modulation.

In a preferred embodiment of this aspect of the invention, the protein kinase is any one of DNA-PK, ATR, ATM or FRAP and the method is for modulating cell cycle checkpoint control.

In a further preferred embodiment the protein kinase is ATM or ATR. Modulators of ATM or ATR, as identified herein, may be useful for treating any of a variety of purposes such as in therapy of ataxia-telangiectasia (AT); modulation of the immune system (ATM appears to be required for the generation of a fully functional immune system); modulating teleomere length (cells of A-T patients lose their telomeres more quickly than normal individuals)—this may be useful ageing, AIDS and other conditions, tumour/cancer therapy; and in augmenting of cancer radiotherapy or chemotherapy.

The aforementioned compounds of the invention or a formulation thereof may be administered by any conventional method including oral and parenteral (eg subcutaneous or intramuscular) injection. The treatment may consist of a single dose or a plurality of doses over a period of time.

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

A seventeenth aspect of the invention provides a method of determining whether an individual has or is predisposed to a defect in DNA repair or cell cycle checkpoint control, the method comprising the steps of (1) obtaining a sample from the patient, (2) determining the concentration of, or subcellular localisation of, inositol hexakisphosphate (IP₆) or other stimulatory inositol phosphate in the sample, and (3) comparing the result with a standard.

The sample obtained from the patient may be any suitable sample. Typically the sample is a sample of a tissue in which DNA repair or cell cycle checkpoint control defects are known to occur, such as skin and in tumour samples. The concentration, or subcellular localisation, of IP₆ or other stimulatory inositol phosphate may be determined by any suitable method. For example, anti-IP₆ antibodies may be made using methods well known in the art which may be used to quantify IP₆, and also to detect its sub cellular location, for example by using suitable staining and microscopic techniques such as confocal microscopy, immunofluorescence microscopy and by immunohistochemistry. Alternatively, IP₆ and other stimulatory inositol phosphates as defined can be measure by suitable analytical techniques such as ELISA. Subcellular localalisation can be compared, for example, by measuring IP₆ in a nuclear extract compared to a cytoplasmic extract.

The invention may also be considered to relate to a method of modulating DNA repair activity comprising performing DNA repair in the presence of an effective modulating amount of an inositol phosphate, or isomer thereof. Preferably, the inositol phosphate is IP₆.

As used herein, when reference is made to any inositol phosphate it includes reference to a salt thereof, particularly a physiologically acceptable salt.

The invention will now be described in more detail by reference to the following Example and Figures wherein:

FIG. 1. Repair of double-strand breaks by non-homologous end-joining. In this schematic model DSBs, caused by either irradiation or chemical assault are bound by the Ku heterodimer (Ku70/80) and the catalytic subunit of the DNA-dependent protein kinase, DNA-PK_{cs}. Binding protects the free ends from nuclease attack while simultaneously initiating the assembly of the NHEJ apparatus. Through an as yet undefined process, DNA ends are bridged, and the XRCC4/DNA ligase IV complex is recruited to the DSB where it effects repair.

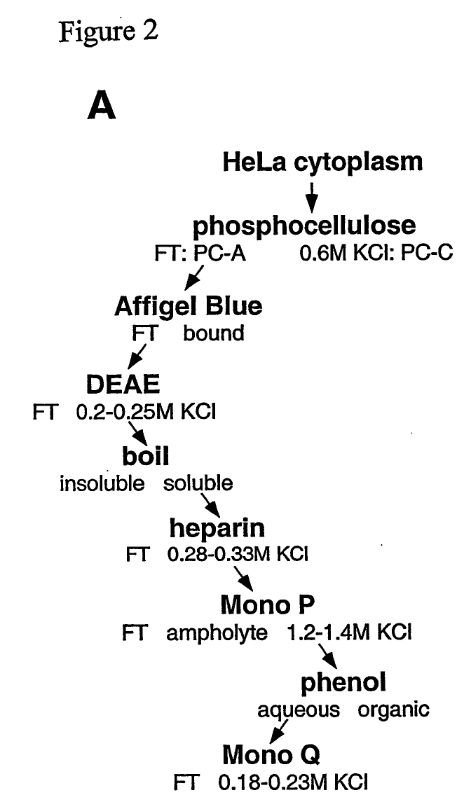

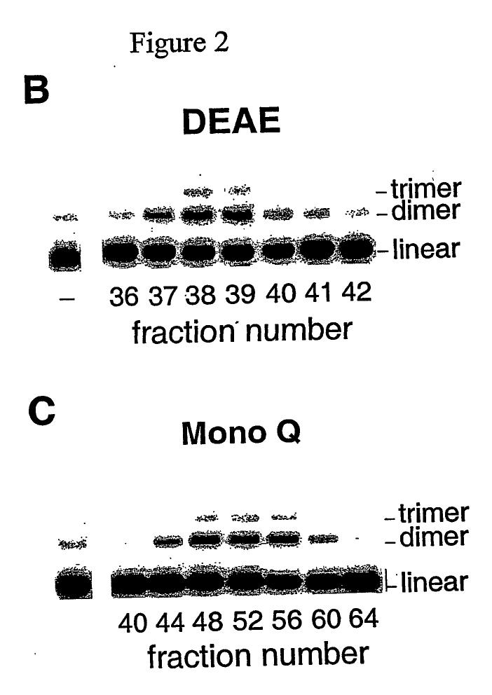

FIG. 2. Purification of SFA. A. Schematic representation of the chromatographic steps taken to purify SFA from HeLa cytoplasmic extracts. B. Complementation of DNA end-joining by the addition of undiluted DEAE fractions to PC-C. Samples were analyzed by gel electrophoresis and ³²P-labeled DNA visualized by autoradiography. C. Fractions eluting from mono Q were diluted 1:50 in L buffer and assayed for the ability to complement end-joining by PC-C. End-joining by PC-C alone (−), and a selected region of each column elution profile is shown. Mobilities of linear, dimer and trimer DNA species are indicated.

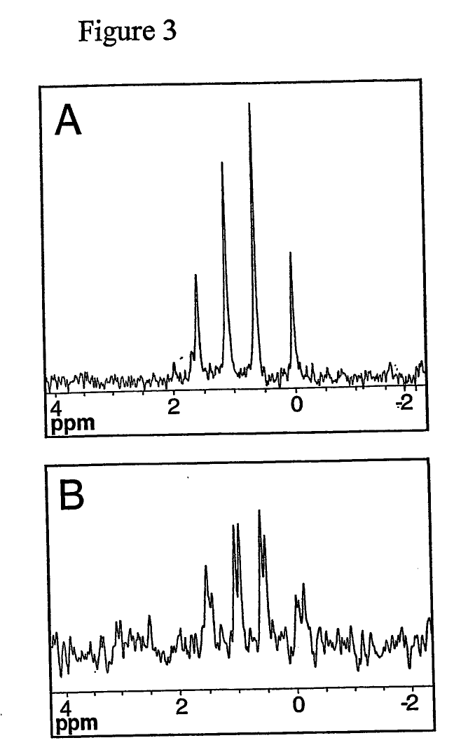

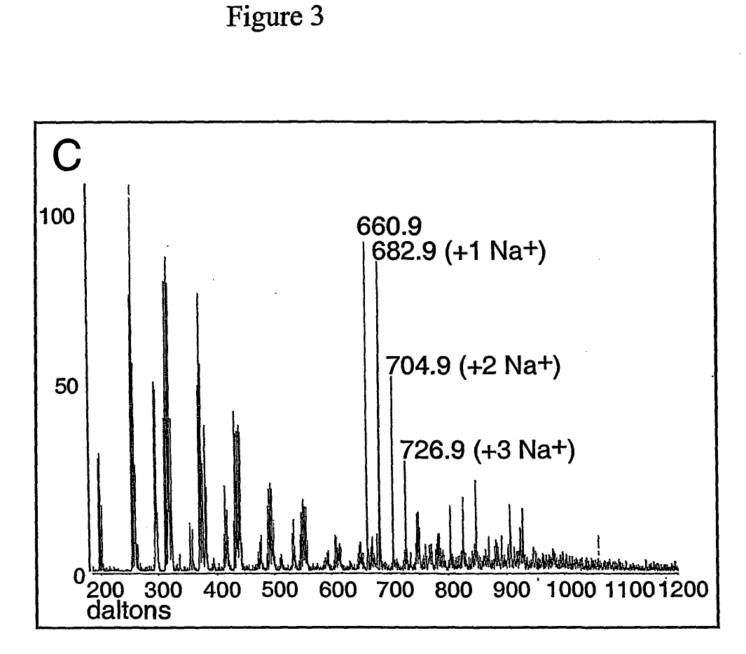

FIG. 3. Physical characteristics of SFA. A. Proton decoupled phosphorus spectra revealed four peaks (ratio 1:2:2:1) close to the phosphoric acid reference at 1.4909 ppm (intensity 2.582), 1.0003 ppm (intensity 5.465), 0.5554 ppm (intensity 6.002) and −0.0850 ppm (intensity 2.927), suggesting phosphate groups and showing no evidence of phosphorus-phosphorus coupling. B. Proton phosphorus coupled spectrum revealed phosphorus proton doublets consistent with the phosphorus being linked to a carbon hydrogen bond. C. Ion-trap spectrum revealed a mass of 660.9 Da, which represents the [mass +1]⁺¹-ion or [659.9+1]⁺¹-ion followed by a series of related sodium salts at 682.9 Da (+1Na⁺), 704.9 Da (+2Na⁺), 726.9 Da (+3Na⁺).

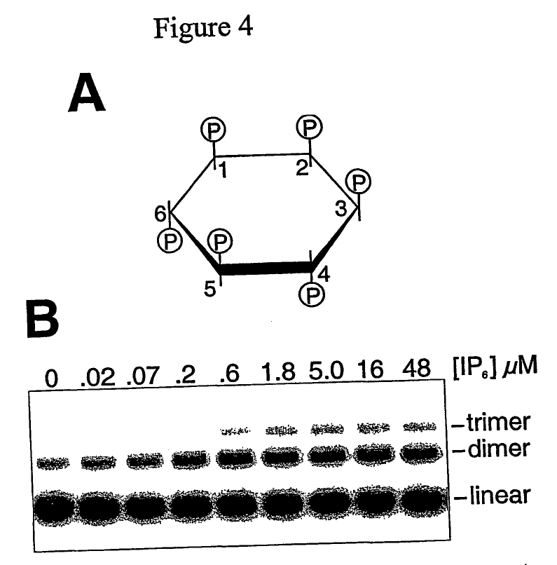

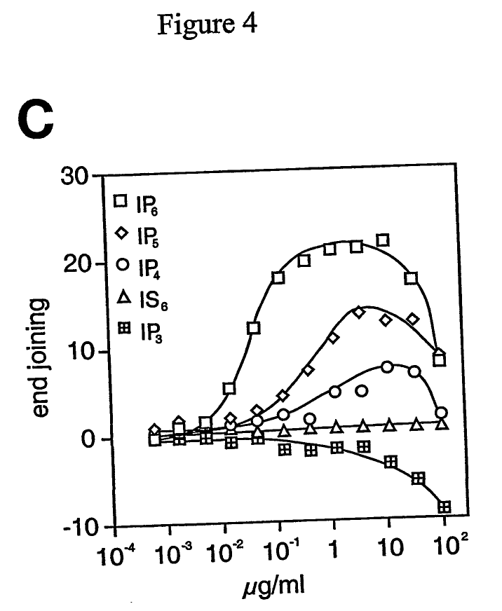

FIG. 4. Stimulation of DNA-PK dependent end-joining by inositol phosphates. A. Schematic representation of IP₆. B. Complementation of PC-C by IP₆. End-joining assays were carried out using PC-C complemented with increasing amounts of IP₆. C. Effect of inositol phosphates on DNA-PK dependent NHEJ. Inositol hexakisphosphate (IP₆), inositol pentakisphosphate (IP₅), inositol tetrakisphosphate (IP₄), inositol trisphosphate (IP₃) or inositol hexasulphate (IP₆) were assayed for their ability to stimulate DNA end-joining by PC-C.

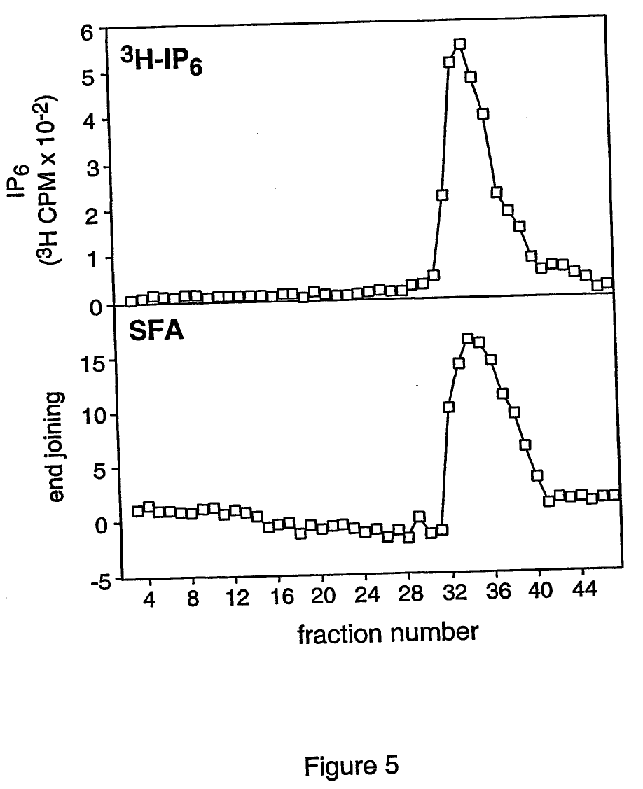

FIG. 5. SFA and IP₆ co-fractionate by strong anion exchange chromatography. A trace amount (4 nM) of 3H-IP₆ was added to a 1 ml aliquot of SFA. The resulting sample was applied to AG 1-X8 resin and eluted as described in Materials and Methods. Top, elution profile of ³H-IP₆ as measured by scintillation counting. Bottom, elution profile of SFA, determined by the complementation of PC-C mediated end-joining.

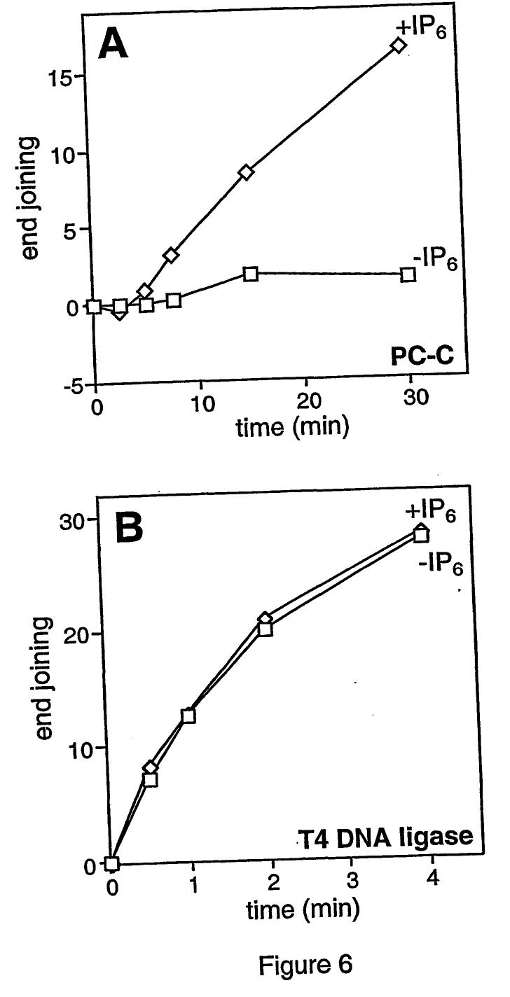

FIG. 6. Specificity of IP₆ for DNA-PK mediated end-to-end ligation. A. DNA end-joining reactions catalyzed by PC-C were analyzed in the presence or absence of 2 μM IP₆. B. Similar reactions carried out using T4 DNA ligase (0.1 u/μl) in place of PC-C.

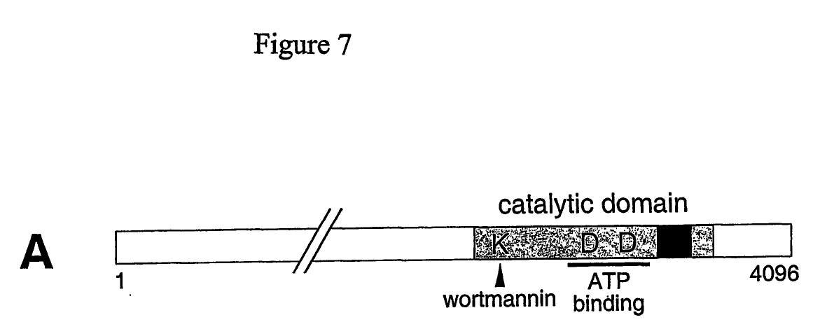

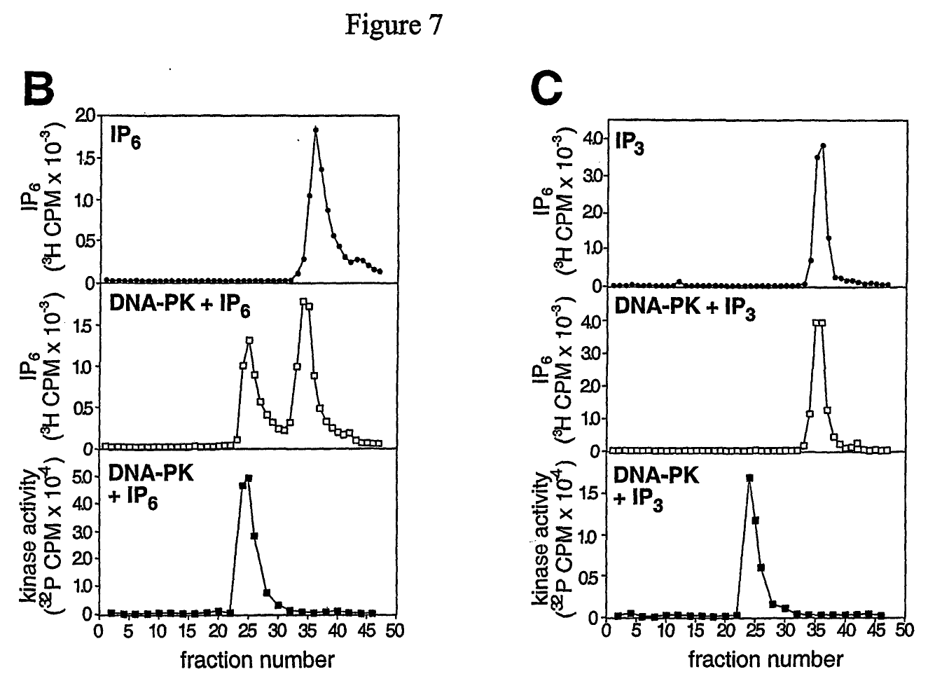

FIG. 7. Binding of IP₆ by DNA-PK. A. Schematic representation of DNA-PKcs. The grey bar represents the C-terminal 380aa which share sequence similarity to the catalytic domain of the PI 3-kinases (Hartley et al, 1995) and the black box indicates the location of the putative inositol phosphate headgroup binding domain of the PI 3-kinases (Wymann and Pirola, 1998). The lysine (K) residue believed to be the target of wortmannin interaction is shown as are the two aspartate (D) residues believed to be located in the ATP binding active site of the protein kinase. B. Gel filtration analysis of ³H-IP₆ binding by DNA-PK. Top, elution profile of ³H-IP₆ in the presence of non-specific proteins (1.8 mg/ml). Middle, elution profile of ³H-IP₆ in the presence of DNA-PK. Bottom, elution profile of DNA-PK kinase activity of fractions shown in B, middle. C. Gel filtration analysis of ³H-IP₃ binding by DNA-PK. Top, elution profile of ³H-IP₃ with protein size standards (1.8 mg/ml). Middle, elution profile of ³H-IP₃ with DNA-PK. Bottom, elution profile of DNA-PK kinase activity of fractions shown in C, middle.

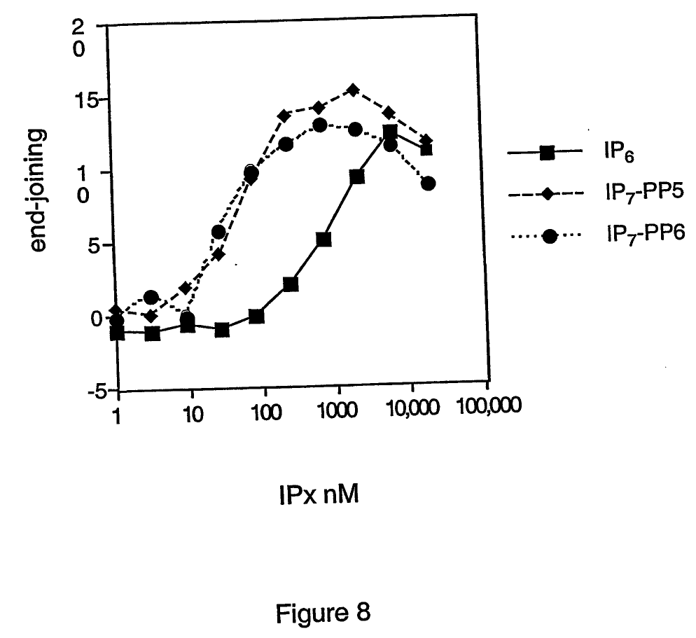

FIG. 8 shows that both IP₇ pp5 and IP₇-pp6 stimulate NHEJ by PC-C and that this stimulation is approximately 10-fold better than that achieved by IP₆.

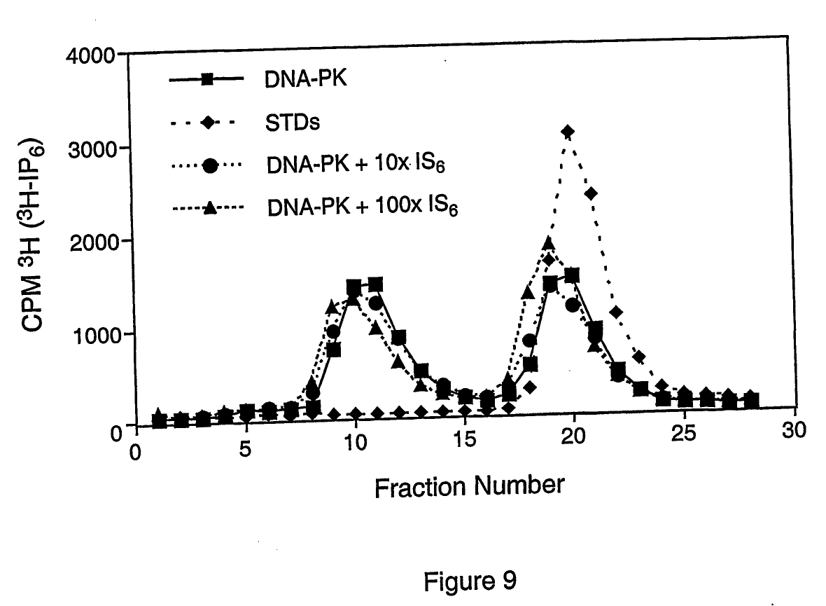

FIG. 9 shows that IS₆ does not compete with IP₆ in binding to DNA-PK.

FIG. 10 shows the distribution of 3H-IP₆ along a gel filtration column under various conditions. Superdex 200 gel filtration, 50 μl fractions, scint. counting 20 μl/fraction 100 nM ³H-IP₆, 100 nM Ku, 100 nM DNA (200 μM DNA ends), 100 nM PK_{cs}. Running buffer. 50 mM HEPES, pH 8.0, 40 mM KOAc, 10% Glycerol, 0.1 M KCl, 1 mM DTT.

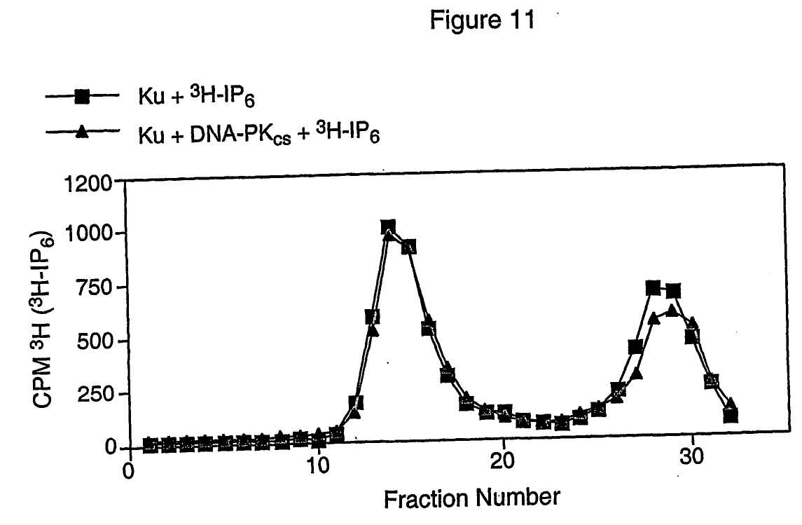

FIG. 11 is an overlay of the Ku+³H-IP₆ and Ku+DNA-PK_{cs}+³H-IP₆ curves from FIG. 10 which emphasises that the presence of DNA-PK_{cs} does not alter the mobility of the Ku-³H-IP₆ curves from FIG. 10 which emphasises that the presence of DNA-PK_{cs} does not alter the mobility of the Ku-³H-IP₆ complex along the gel filtration column.

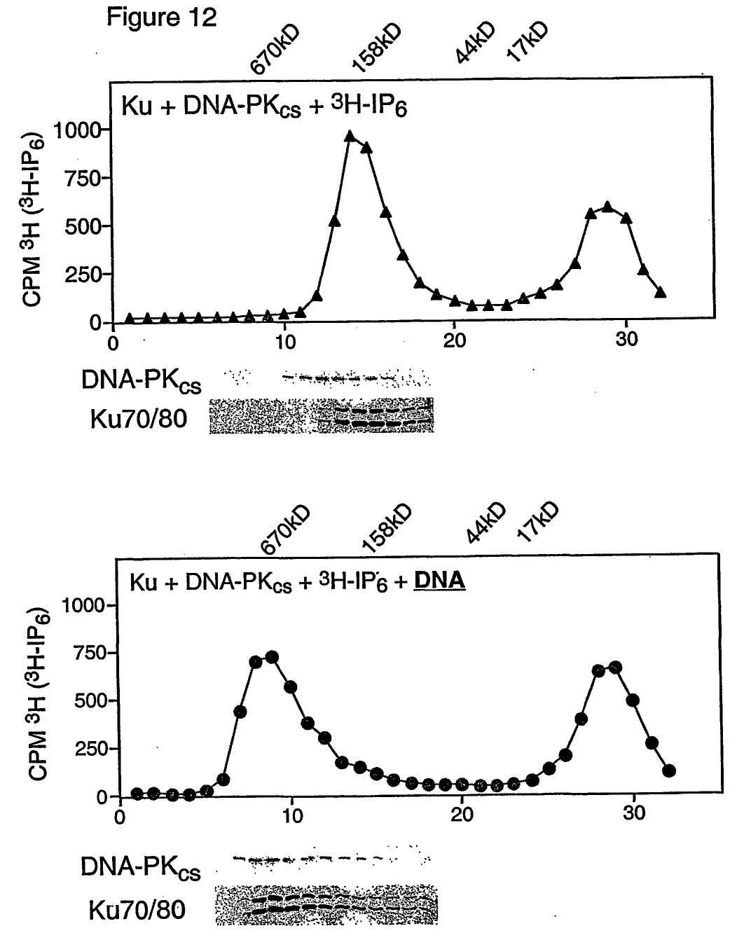

FIG. 12 shows the result of gel filtration carried out on DNA-PK+³H-IP₆ in the presence or absence of DNA.

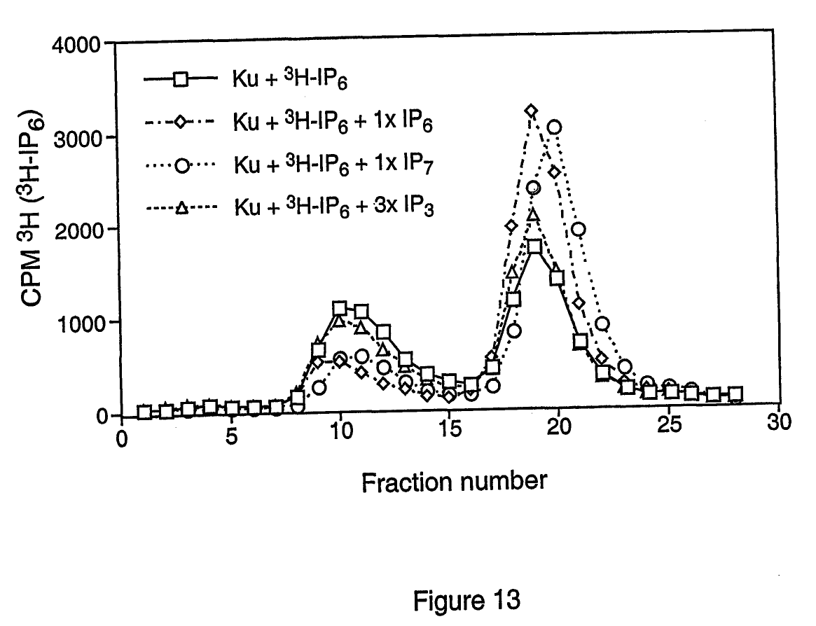

FIG. 13 shows the result of competition analysis of IP₆ binding by Ku using IP₃, IP₆ and IP₇.

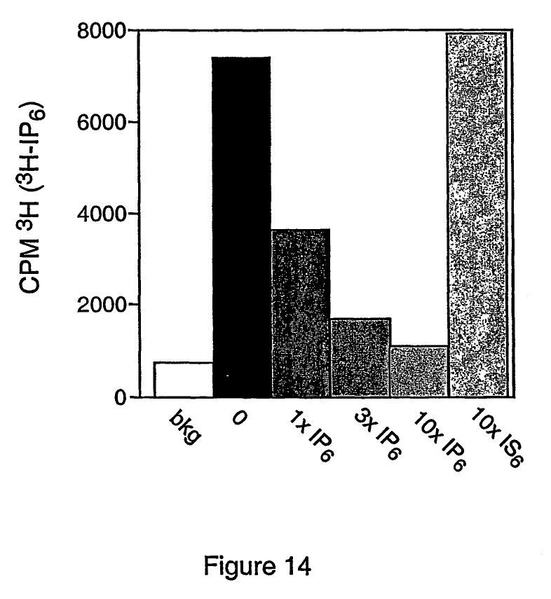

FIG. 14 shows the results of specificity trials using the spin column method.

### EXAMPLE 1

### Stimulation of DNA-PK Dependent Non-Homologous End-Joining by Inositol Phosphate

Results

Purification of a Factor that Stimulates DNA End-Joining in Vitro

In previous studies, cell-free extracts capable of promoting DNA end-joining were fractionated by phosphocellulose chromatography (Baumann and West, 1998). One fraction (designated PC-C), which contained all components known to be required for NHEJ in vivo (Ku70/80, DNA-PK_{cs}, XRCC4, DNA ligase IV), showed only limited ability to join DNA ends in vitro. The ability to promote end-joining, however, could be restored by addition of a second fraction (designated PC-A). This complementation assay provided the basis for the purification of the stimulatory factor in PC-A, which we now designate Stimulatory Factor A (SFA).

Subcellular fractionation of HeLa cells into nuclear and cytoplasmic fractions, showed the cytoplasm to be rich in SFA (data not shown). By comparison, relatively low levels of SFA were detected in nuclear extracts. The presence of sheared chromosomal DNA in the nuclear extracts, which could compete for the factors involved in NHEJ, however, made it difficult to accurately assess the relative levels of SFA in these two subcellular compartments. Given that the removal of nuclei would minimize the amount of contaminating DNA, we chose to prepare SFA from the cytoplasmic fraction of 300 L of HeLa cells.

Preliminary binding trials showed that cation-exchange resins such as mono S or SP-sepharose failed to bind SFA. Therefore, anion-exchange resins of various strengths were used throughout the purification scheme (FIG. 2). During purification, we were somewhat surprised to discover that SFA was heat-stable, insensitive to treatment with phenol, and insoluble in CHCl₃. UV absorbance spectroscopy demonstrated that a sample of concentrated SPA did not absorb at 280 nm, indicating a lack of aromatic amino acids (data not shown). All attempts to degrade SPA using proteases (trypsin, V8 protease and proteinase K+SDS) failed (data not shown). Taken together, these observations suggest that SFA, previously assumed to be a protein participant in NHEJ, is not a polypeptide.

To investigate the possibility that SFA might be a nucleic acid (RNA or DNA), SFA was treated with either RNAse A, NaOH (0.1-1.0 M at 60° C.), DNAse I or micrococcal nuclease. These treatments had no effect on the ability of SFA to stimulate end-joining (data not shown). UV absorbance spectroscopy demonstrated that a sample of concentrated SFA did not absorb at 260 μm, indicating a lack of purine or pyrimidine moieties in the sample (data not shown). These data demonstrate that SFA is not a nucleic acid.

Chemical Analysis of SFA

To identify the active component in SFA, the sample was subjected to NMR and mass spectroscopy. Proton decoupled phosphorus NMR spectra (FIG. 3A) revealed four peaks (ratio 1:2:2:1) close to the phosphoric acid standard suggesting six phosphate groups. The 1:2:2:1 ratio of peak intensities suggests two independent sets of equivalent phosphates as well as two nonequivalent individual phosphates. Proton phosphorus coupled spectra (FIG. 3B) revealed phosphorus proton doublets, consistent with each of the phosphate groups being linked to a carbon participating in a carbon-hydrogen bond. These data suggest that the SFA sample contains an organophosphorus compound containing 6 phosphates, each directly linked to a (—CH) group.

The molecular mass of SFA was determined by mass spectroscopy (FIG. 3C). While no significant signal was observed in the range commonly associated with macromolecules, polypeptides or polymeric nucleic acids, the SFA sample was found to contain a number of species of low molecular mass. Although the SFA sample was found to be heterogeneous, a clear peak was detected at a mass of 660.9 Da. Additionally, an array of peaks which differed from the original 660.9 Da peak by 22 Da (the mass of sodium Na⁺) were observed downstream of the 660.9 Da peak. These masses appear to correspond to the +1 (Na), +2 (Na⁺) and +3 (Na⁺) salts of the 660.9 Da species.

Identification of the Active Component of SFA as IP₆

Inositol is a fully hydroxylated six-carbon ring which is found in a number of phorphorylation states ranging from mono- through hexakisphosphate. Inositol hexakisphosphate (IP₆) (FIG. 4A) shares the same molecular weight as SFA (659.9 Da) and has the same phosphorus content The presence of six phosphates in IP₆ and their conformation in aqueous solution (Barrientos and Murthy, 1996; Costello et at, 1976; Emsley and Niazi, 1981) is in keeping with the obtained proton-decoupled ³¹P NMR spectra of SFA (FIG. 3A) which matches the proton-decoupled ³¹p spectrum for the mono-ionic form of IP₆. Furthermore, each phosphate group of IP₆ is linked to a carbon participating in a carbon-hydrogen bond, which is in accord with the proton coupled phosphorus NMR spectum (FIG. 3B). These results indicate that the 660.9 Da species detected by mass spectroscopy is the protonated form of IP₆ ([659.9+1]+1 Da), and that the above described array of peaks represent the sodium salts that would readily form with IP₆. To confirm that the active component in SFA is indeed IP⁶, commercially available IP₆ was assayed for its ability to stimulated end-joining by PC-C. As shown in FIG. 4B, IP₆ stimulated end-joining at concentrations in the region of 100 nM and stimulation was maximal at 1 μM.

To assess the specificity of NHEJ for IP₆, we compared the ability of IP₆ to stimulate end-joining with other inositol phosphates (IP₅, IP₄ and IP₃). In addition, inositol hexasulphate (IS₆)—an inositol compound which would provide a charge distribution similar to that of IP₆, while presenting sulfate rather than phosphate groups—was also assayed. It was found that IS₆ was unable to stimulate end-joining, demonstrating a clear requirement for phosphate groups (FIG. 4C). Indeed, we found that IP₆ proved to be the most effective inositol phosphate compound of those tested. IP₅ and IP₄ were also able to stimulate end-joining, but the efficiency of this stimulation was reduced relative to IP₆. These data show that end-joining requires a phosphorylated inositol species, and that the stimulation of NHEJ is directly related to the extent of phosphorylation.

Further evidence that IP₆ is the active component in SFA was obtained by strong anion exchange (SAX) chromatography, using a resin that is commonly utilized to separate highly charged molecules such as the inositol phosphates. To determine whether the NHEJ stimulating activity in SFA co-fractionated with IP₆, an aliquot of SFA was spiked with a trace amount of ³H-IP₆ (4 nM), and the mixture was chromatographed on AG 1-X8 resin. Complementation assays were performed to detect the presence of SFA, and the ³H-IP₆ content was assessed by scintillation counting. As shown in FIG. 5, the peaks of SFA and ³H-IP₆ were coincident

Although IP₆ is of small molecular size (660 Da), its high charge to mass ratio and the hydrogen bonding observed between phosphate groups (Emsley and Niazi, 1981) results in a larger apparent molecular size in aqueous solutions at low ionic strength. This has been observed by the retention of IP₆ by dialysis membranes at low ionic strength, and the passage of IP₆ through the same membrane at high ionic strength (data not shown). Equilibrium dialysis trials were performed to compare the movement of SFA and ³H-IP₆ across a dialysis membrane (12-14 kDa cutoff). Both SFA and ³H-IP₆ were retained during equilibrium dialysis at low ionic strength, and at high ionic strength both SFA and ³H-IP₆ passed through the dialysis membrane (data not shown). These observations add to the list of physical properties shared by SFA and IP₆.

Specificity of IP₆ for DNA-PK Dependent End-Joining

The data presented above show that the stimulatory factor purified from HeLa cells is inositol hexakisphosphate (IP₆). Because many substances (eg PEG, PVA) have non-specific stimulatory effects on the efficiency of DNA ligases in vitro, we next determined whether IP₆ was specific for NHEJ mediated by DNA-PK. To do this, we compared the ligation efficiencies of PC-C and T4 DNA ligase in the presence or absence of IP₆. It was found that IP₆ stimulated end-joining by PC-C, whereas the efficiency of ligation by T4 DNA ligase was unaffected (FIG. 6).

Binding of ³H-IP₆ by DNA-PK

The catalytic domain of DNA-PK_{cs} is related to that found in the PI 3-kinases which phosphorylate inositol phospholipids. Previous studies have shown that in the PI 3-kinases recognition of the inositol phosphate headgroup is mediated by defined sequences within this conserved domain (FIG. 7A) (Bondeva et al, 1998). Given that NHEJ is dependent upon DNA-PK and is stimulated by the addition of IP₆ it was plausible that this stimulatory effect is due to a physical interaction between IP₆ and DNA-PK.

Gel filtration analysis using ³H-IP₆ and purified DNA-PK was carried out to assess the ability of DNA-PK to interact with IP₆. As shown in FIG. 7B ³H-IP₆ was retained by the gel filtration media in the absence of DNA-PK and observed in fractions late in the elution profile. In this experiment the retention of ³H-IP₆ was observed in the presence of protein size standards which serve to establish the quality of the elution profile and provide a selection non-specific proteins which have no effect on the mobility of 3H-IP₆. In the presence of DNA-PK ³H-IP₆ was also observed in early fractions of the elution profile which correspond to the peak of DNA-PK activity. The altered retention of ³H-IP₆ by gel filtration media in the presence of DNA-PK and the cofractionation of ³H-IP₆ and DNA-PK activity indicate that DNA-PK is capable of binding IP₆.

To assess the specificity of IP₆ binding by DNA-PK we examined the ability of DNA-PK to bind another inositol phosphate. Given that IP₃ does not stimulate NHEJ (FIG. 4C) we selected ³H-IP₃ for this comparison. In contrast with ³H-IP₆, the retention of ³H-IP₃ by the gel filtration matrix is not altered by the presence of DNA-PK (FIG. 7C). Furthermore, the fractions which show substantial DNA-PK activity contain only background levels of ³H-IP₃. This argues against the possibility that the observed binding of IP₆ by DNA-PK (FIG. 7B) might be due to non-specific interactions between DNA-PK and the multiple phosphate groups of IP₆. Taken together the data presented in FIG. 7 demonstrate a physical interaction between DNA-PK and IP₆ which does not extend to all of the inositol phosphates.

Discussion

We have isolated a factor that stimulates NHEJ in vitro, and have identified this factor as inositol hexakisphosphate (IP₆). What is the role of IP₆ in NHEJ? IP₆ has been shown to function as a phosphatase inhibitor at concentrations of 10 μM or greater (Larsson et al, 1997). Given that IP₆ is capable of stimulating NHEJ at 1% of this concentration it is unlikely that IP₆ functions in NHEJ as an inhibitor of phosphatases. The inability of IP₆ to enhance end-joining by T4 DNA ligase argues against the possibility that IP₆ stimulates NHEJ via a non-specific mechanism such as macromolecular crowding by volume exclusion. More likely, the low concentration at which IP₆ can stimulate end-joining (100 μM) indicates that stimulation results from the interaction of IP₆, either directly or indirectly, with some component of the end-joining apparatus.

The ability to stimulate NHEJ does not appear to be restricted to IP₆, because we found that IP₅ and IP₄ were also able to stimulate end-joining, though to a lesser extent. Indeed, the efficiency of stimulation appears to be directly proportional to the degree of phosphorylation of the inositol phosphate. This correlation fits a model in which IP₆ functions as a ligand and is bound by a ligand binding species. In such a model, small alterations in the structure of the ligand (in this case changes from IP₆ to IP₅ or to IP₄) would be predicted to result in incremental decreases in the affinity or stability of the ligand binding interaction. If ligand binding is essential for end-joining, then these small alterations in ligand structure would be apparent as incremental decreases in end-joining efficiency. The important prediction made by this model is that a species which both binds IP₆ and participates in NHEJ must be present in the PC-C fraction. We have identified this IP₆ binding species as DNA-PK.

The apparent requirement for IP₆ in NHEJ suggests that DNA-PK is in some way NHEJ-inactive in the unbound state, and that there is a transition upon ligand (IP₆) binding resulting in an NHEJ-active species. Additionally, binding of IP₆ may influence the ability of DNA-PK to interact with other components of the NHEJ apparatus.

The consequences of IP₆ binding by DNA-PK might be structural in nature, possibly due to an allosteric shift upon association with IP₆. Alternatively, binding of IP₆ could simply alter the surface charge distribution of DNAPK Such an alteration of local electrostatic potential has been observed in the binding of IP₆ to phosphoglycerate mutase (Rigden et al, 1999). In this case, ligand binding was mediated by both hydrogen bonding interactions and by the strong positive electrostatic potential of the active site cleft. Occupancy of this highly charged cleft by IP₆ exposes several phosphates to the solvent, which has a pronounced effect on the local electrostatic potential relative to the unbound state. An alteration of this kind would result in a more passive transition from the NHEJ-inactive to the NHEJ-active state of DNA-PK. Of course these two possibilities are not mutually exclusive and both mechanisms might influence the substrate specificity of DNA-PK as well as its potential to participate in extensive protein complexes.

As far as we are aware, DNA-PK is the only protein known to participate in NHEJ that has been demonstrated to bind IP₆. DNA-PK is a member of the phosphatidylinositol 3-kinase (PI3K)-related kinase family, as are ATM and ATR. All three proteins exhibit a strong sequence homology to the PI 3-kinases, especially in the catalytic core domain that binds and phosphorylates the phosphoinositol headgroup of phosphatidylinositol. However, no phosphorylation of lipid substrates has been observed by DNA-PK_{cs}, or by other members of this PI3K-related family of kinases (Carpenter and Cantley, 1996; Hunter, 1995; Keith and Schreiber, 1995; Wymann and Pirola, 1998).

It is tempting to postulate that the PI3K-related kinases are derived from a common ancestor that possessed both protein kinase and lipid kinase functions. Mutation of the PI3K catalytic domain could result in loss of lipid kinase activity, while retaining an affinity for the phosphoinositol headgroup. This hypothesis is supported by the work by Bondeva et al (Bondeva et al, 1998) demonstrating that substitution of the putative phosphoinositol headgroup interaction site of PI3Kγ can alter or abort lipid kinase activity in vitro, while leaving protein kinase function unaffected. Clearly an important next step in the investigation of both NHEJ and the PI3K-related kinases will be to characterize the interaction between DNA-PK and IP₆ and to examine the consequences of this binding event

Experimental Procedures

Materials

myo-inositol 1,4,5-trisphosphate (IP₃), D-myo-inositol 1,3,4,5-tetrakisphosphate (IP₄), myo-inositol 1,3,4,5,6-pentakisphosphate (IP₅) and inositol hexasulphate (IS₆) were purchased from Sigma. myo-inositol 1,2,3,4,5,6-hexakisphosphate (IP₆) was purchased from Sigma and also from Calbiochem. ³H-IP₆ (10-30 Ci/mmol) was purchased from NEN.

Preparation of PC-C and Complementation Assay

HeLa whole cell extracts were prepared and fractionated step-wise over phosphocellulose as described (Baumann and West, 1998). Fraction PC-C was dialyzed for 2 hours against L buffer (20 mM Tris-HCl pH 8.0, 25 mM KOAc, 0.5 mM EDTA, 10% glycerol, 1 mM DTT) and stored at −80° C.

End joining reactions (10 μl) were carried out in 50 mM HEPES pH 8.0, 40 mM KOAc, 0.5 mM Mg(OAc)₂, 1 mM ATP, 1 mM DTT, 0.1 mg/ml BSA, contained 2-3 μl (3-5 μg) of PC-C and HindIII-linearized 5′-³²P-labeled pDEA7Z DNA (10 ng). Incubation was for 1 hour at 37° C. ³²P-labeled DNA products were deproteinized and analyzed by electrophoresis through 0.6% agarose gels followed by autoradiography. Quantification of joining efficiency was carried out by phosphorimaging and the data are presented as (% total ends-joinedₛₐₘₚₗₑ)−(% total ends-joined_{PC-C}) unless otherwise stated. For assays of inositol phosphates, aqueous solutions were added directly to the end-joining reactions. For the screening of column fractions; 0.5-1 μl of diluted (in L buffer) or undiluted column fractions were added

Preparation of Cytoplasmic Extracts

300 L of HeLa cells were cultured in suspension to a density of 5×10⁵ cells/ml, harvested by centrifugation, washed twice with PBS, flash-frozen on liquid nitrogen and stored at −80° C. When required, 2 packed cell volumes of hypotonic lysis buffer (10 mM Tris-HCl pH 8.0, 1 mM EDTA, 1 mM DTT) was added and the cells were held on ice for 20 min. The cells were opened by dounce homogenization (35 strokes with a “B” pestle) in the presence of protease inhibitors (1 mM PMSF, 2.2 ng/ml aprotinin, 1 ng/ml leupeptin, 1 ng/ml pepstatin A and 1 ng/ml chymostatin). The resulting homogenate was centrifuged for 30 minutes at 10,000 rpm to pellet membraneous cellular debris and intact nuclei. The resulting cytoplasmic fraction was dialyzed for 2 hours at 4° C. against L buffer and then centrifuged for 45 min at 45,000 rpm at 4° C. in a Beckman Ti45 rotor. All dialysis steps were carried out in tubing with a molecular weight cutoff of 12-14 kDa

Purification of SFA

Cytoplasmic extracts (from the equivalent of 100 L cultured cells) were first batch fractionated by absorption to ⅓ vol phosphocellulose (Whatman P-11) by gently rocking for 30 min at 4° C. Following centrifugation, the unbound fraction was reserved. The resin was washed twice with an equal volume of L buffer. The unbound fraction and the 2 washes were pooled, and spun for 45 min at 45,000 rpm at 4° C. in a Beckman Ti45 rotor. The supernatant was then loaded onto a 5×10 cm phosphocellulose column equilibrated in L buffer. The flow through passed directly onto a 5×10 cm affigel blue column (Bio-Rad) equilibrated in the same buffer. The flow through was loaded onto a 1.6×39 cm DEAE Fast Flow column (Pharmacia) equilibrated in L buffer, which was eluted with 15 column volumes of a 0-0.5 M KCl linear gradient in L buffer. Active fractions eluting between 0.2-0.25 M KCl were pooled (designated DEAE SFA) and stored at −80° C. until all 300 L equivalent of HeLa cell cytoplasm had been fractionated.

DEAE-SFA fractions were heat denatured by boiling for 15-20 minutes, then centrifuged for 45 min at 45,000 rpm at 4° C. in a Beckman Ti45 rotor to remove insoluble aggregates. The supernatant was dialyzed for 6 hours against L buffer, applied to a 2.6×37 cm affi-gel heparin column (Bio-Rad) equilibrated in the same buffer, then eluted with a 15 column volume 0-1.0 M KCl linear gradient in L buffer. Active fractions eluting between 0.280.33 M KCl were pooled, dialyzed for 6 hours against L buffer, then loaded onto a Mono P Hr 5/20 column (Pharmacia). The column was washed with 5 column volumes of L buffer, then with 14 volumes of polybuffer 74-HCl (pH 4.0) and finally eluted with a 50 column volume 0-2.0 M KCl linear gradient Active fractions eluting between 1.2-1.35 M KCl were pooled, extracted with phenol/CHCl₃, dialyzed for 6 hours against L buffer, loaded onto a Mono Q Hr 5/5 column (Pharmacia) equilibrated in L buffer, and then eluted with a 30 column volume 0-1.0 M KCl linear gradient in L buffer. Active fractions eluting from the column between 0.18-0.23 M KCl were pooled, dialyzed overnight at 4° C. against dH₂O and stored at −20° C.

Strong Anion Exchange Chromatography

AG 1-X8 resin (200-400 mesh, formate form, Bio-Rad) was washed with 5 volumes of 1.0 M formic acid, then with 15 column volumes of dH₂O. SFA was dialyzed against dH₂O overnight at 4° C. A trace (4 nM) amount of ³H-IP₆ was added, and the sample was applied to a 1.0×1.3 cm AG 1-X8 column equilibrated with dH₂O. The column was washed with 10 column volumes of dH₂O, then eluted with a 30 column volume 0-2.75 M ammonium formate linear gradient in 0.1 M formic acid.

Mass Spectrometry

SFA sodium salt was acidified with 1M HCl to approximately pH 2.0, diluted with 1 equivalent of 70% methanol/30% formic acid and a 10 μl sample was loaded into a carbon coated nano spray capillary needle (Protana) and the molecular weight determined by mass spectrometry using an ion-trap (LCQ Thermoquest).

NMR

SFA was lyophilized and resuspended in D₂O. Measurements were carried out using a Bruker AC-300 pulse Fourier transform NMR spectrometer operating at 300.13 Mhz for protons ³¹P spectroscopy (5 mm sample in 10 mm broad band probe), and at 121.497 Mhz with broad band proton decoupling (composite pulse). Reference ³¹P signals were detected in a +/−50 ppm window.

Equilibrium Dialysis

For analysis at low ionic strength, samples (1 ml) were dialyzed against 0.5 L dH₂O for 21 hours at 4° C. with sting. For high ionic strength, samples were dialyzed against 0.25 L of 2.5 M NaCl for 48 hours at 4° C. Dialysis tubing with a molecular weight cutoff of 12-14 kDa was used.

T4 DNA Ligation Assay

Reactions (10 μl) containing T4 DNA ligase (NEB) were carried out in 1×T4 DNA ligase buffer (NEB) at 25° C. in the presence and absence of inositol phosphates. Reactions were stopped, deproteinized and the products analyzed by agarose gel electrophoresis.

Gel Filtration Analysis of ³H-IP₆ Binding by DNA-PK

Binding reactions (55 μl) were carried out in 25 mM HEPES pH 7.5, 50 mM KCl, 10 mM MgCl₂, 1 mM DTT, 10% glycerol, 0.1% NP-40 with 5000 units of DNA-PK (Promega) or 1.8 mg/ml of protein size standards for gel filtration (BioRad) and 100 nM ³H-IP₆ or ³H-IP₃ at 4° C. for 30 min. Complexes were resolved on a Superose 12 PC3.2/30 column run in 50 mM HEPES pH 8.0, 40 nM KOAc, 0.1M KCl, 10% glycerol, 1 mM DTT at 40 μl/min. 50 μl fractions were collected; 20 μl samples of each fraction were used for ³H scintilation counting (10 min/sample in 5 ml EcoscintA) and 10 μl were assayed for DNA-PK kinase activity using the SignaTECT DNA-PK assay system (Promega).

### REFERENCES FOR EXAMPLE 1

Barrientos, L. G., and Murthy, P. P. N. (1996). Conformational studies of myo-inositol phosphates. Carbohydr. Res. 296, 39-54.

Baumann, P., and West, S. C. (1998). DNA end-joining catalyzed by human cell-free extracts. Proc. Natl. Acad. Sci. USA. 95, 14066-14070.

Blunt, T., Finnie, N. J., Taccioli, G. E., Smith, G. C. M., Demengeot, J., Gottlieb, T. M., Mizuta, R., Varghese, A. J., Alt, F. W., Jeggo, P. A., and Jackson, S. P. (1995). Defective DNA-dependent protein kinase activity is linked to V(D)J recombination and DNA repair defects associated with the murine scid mutation. Cell 80, 813-823.

Bondeva, T., Pirola, L., Bulgarelli-Leva, G., Rubio, I., Wetzker, R., and Wymann, M. P. (1998). Bifurcation of lipid and protein kinase signals of PI3 K gamma to the protein kinases PKB and MAPK. Science 282, 293-296.

Carpenter, C. L., and Cantley, L. C. (1996). Phosphoinositide kinases. Curr. Opin. Cell. Biol. 2, 153-158.

Chu, G. (1997). Double-strand break repair. J. Biol. Chem. 272, 24097-24100.

Costello, A. J. R., Glonek, T., and Myers, T. C. (1976). ³¹P nuclear magnetic resonance-pH titrations of myo-inositol hexaphosphate. Carbohydr. Res. 46, 159-171.

Critchlow, S. E., and Jackson, S. P. (1998). DNA end-joining: from yeast to man. TIBS 23, 394-398.

Emsley, J., and Niazi, S. (1981). The structure of myo-inositol hexaphosphate in solution: ³¹P N.M.R. investigation. Phosphorus and Sulfur 10, 401-408.

Essers, J., van Steeg, H., de Wit, J., Swagemakers, S. M. A., Vermeij, M., Hoeijmakers, J. H. J., and Kanaar, R. (2000). Homologous and non-homologous recombination differentially affect DNA damage repair in mice. EMBO J. 19, 1703-1710.

Hartley, K. O., Gell, D., Smith, G. C. M., Zhang, H., Divecha, N., Connelly, M. A., Admon, A., Leesmiller, S. P., Anderson, C. W., and Jackson, S. P. (1995). DNA-dependent protein kinase catalytic subunit: a relative of phosphatidylinositol 3-kinase and the ataxia telangiectasia gene product Cell 82, 849-856.

Hunter, T. (1995). When is a lipid kinase not a lipid kinase? When it is a protein kinase. Cell 83, 1-4.

Keith, C. T., and Schreiber, S. L. (1995). PIK-related kinases: DNA repair, recombination, and cell-cycle checkpoints. Science 270, 50-51.

Larsson, O., Barker, C. J., Sjoholm, A., Carlqvist, H., Michell, R. H., Bertorello, A., Nilsson, T., Honkanen, R. E., Mayr, G. W., Zwiller, J., and Berggren, P. O. (1997). Inhibition of phosphatases and increased Ca2+ channel activity by inositol hexakisphosphate. Science 278, 471-474.

Leber, R., Wise, T. W., Mizuta, R., and Meek, K. (1998). The XRCC4 gene product is a target for and interacts with the DNA-dependent protein kinase. J. Biol. Chem. 273, 1794-1801.

Modesti, M., Hesse, J. E., and Gellert, M. (1999). DNA binding of Xrcc4 protein is associated with V(D)J recombination but not with stimulation of DNA ligase IV activity. EMBO J. 18, 2008-2018.

Rigden, D. J., Walter, R. A., Phillips, S. E., and Fothergill-Gilmore, L. A. (1999). Polyanionic inhibitors of phosphoglycerate mutase: combined structural and biochemical analysis. J. Mol. Biol. 289, 691-699.

Smith, G. C. M., and Jackson, S. P. (1999). The DNA-dependent protein kinase. Genes Dev. 13, 916-934.

Taccioli, G. B., Rathbun, G., Oltz, E., Stamato, T., Jeggo, P. A., and Alt, F. W. (1993). Impairment of V(D)J recombination in double-strand break repair mutants. Science 260,207-210.

Takata, M., Sasaki, M. S., Sonoda, E., Morrison, C., Hashimoto, M., Utsumi, H., Yamaguchi-Iwai, Y., Shinohara, A., and Takeda, S. (1998). Homologous recombination and non-homologous end-joining pathways of DNA double-strand break repair have overlapping roles in the maintenance of chromosomal integrity in vertebrate cells. EMBO J. 17, 5497-5508.

Weaver, D. T. (1996). Regulation and repair of double-strand DNA breaks. Crit. Rev. Eukaryot. Gene Expr. 6, 345-375.

Wymann, M. P., and Pirola, L. (1998). Structure and function of phosphoinositide 3-kinases. Biochim. Biophys. Acta 1436, 127-150.

### EXAMPLE 2

### High Throughput Screening Assay for NHEJ

A microtiter-plate (96-wells) assay which would work like an ELISA assay is used. A “targef” duplex DNA is adhered to the plate (call the sequence oligos 1 and 2 which are complementary and have an exposed, unmodified terminus). Cell extracts are mixed with a “detection” duplex DNA (linear duplex—oligos 3 and 4 which are complementary—which have one exposed, unmodified terminus (which will be ligated to the target DNA) and the other terminus is both 5′ and 3′ blocked by biotin; oligo 3 is 5′ biotinylated, oligo 4 is 3′ biotinylated. The biotin moieties serve two functions: 1) block the end of the DNA and prevent additional ligation which would result in multimerization and non-linear report of activity and 2) for detection. NHEJ reaction components, including IP₆, are incubated in the plate (which presents the target duplex) in the presence of the detection DNA (and buffer and Mg⁺⁺ and ATP). The plate is washed to remove proteins and excess detection DNA. The only way for the detection DNA to remain on the plate is by ligation with the target DNA. The presence of the detection duplex indicated NHEJ—this retention should be quantitative. Detection/quantification of the biotin may be carried out as with an ELISA assay—using avidin coupled enzymes and a chromogenic enzyme substrate.

### EXAMPLE 3

### IP₇ Stimulates NHEJ Better than IP₆

IP₆ can be additionally phosphorylated to IP₇. This results in a species that is pyrophosphorylated at one carbon of the inositol ring. We tested IP₇ pp5 and IP₇-pp6 to see if these polyphosphorylated inositols would stimulate end-joining by PC-C. As shown in FIG. 8 both IP₇ species do stimulate NHEJ by PC-C and this stimulation is approximately 10-fold better than that achieved with IP₆. These data suggest that, while IP₆ is highly effective at stimulating NHEJ, IP₆ may not be the only biologically relevant effector of NHEJ among the inositol polyphosphates.

### EXAMPLE 4

### The Ku70/80 Heterodimer of DNA-PK Binds IP₆ with Specificity

As previously described, DNA-PK (composed of the Ku 70/80 heterodimer and PI-3-like kinase DNA-PK_{cs}) binds IP₆ with specificity. We have determined that within this heterodimer the Ku 70/80 heterodimer is responsible for this binding activity. Furthermore, the Ku-IP₆ complex is capable of binding DNA forming a higher-order complex. Finally, a supercomplex can be formed that contains Ku, IP₆, DNA-PK_{cs} and DNA.

The specificity of IP₆ recognition by DNA-PK was assessed by competition trials using inositol hexasulphate (IS₆). As shown in FIG. 9, tritiated IP₆ (³H-IP₆) is not bound by any of the molecular weight standards used to calibrate the gel filtration column (diamonds, STDs) and is detected in the far-included volume which elutes late in the column profile. Addition of DNA-PK results in an increase in the mobility of the ³H-IP₆ indicating complex formation. IS₆, presented in 10- and 100-fold molar excess have no effect on the amount of ³H-IP₆ bound by DNA-PK. These data, when taken together with binding trials using ³H-IP₃, indicate specific binding of IP₆ by DNA-PK

Ku 70/80 and DNA-PK_{cs} were purified to homogeneity and used to examine the binding of IP₆ by DNA-PK ³H-IP₆ was used in these binding experiments and the curves presented in FIG. 10 represent the distribution of ³H-IP₆ along a gel filtration column under various conditions. ³H-IP₆ observed in a peak below 17 kD represents unbound ³H-IP₆. In the presence of purified Ku 70/80 (±DNA-PK_{cs}) ³H-IP₆ elutes early from the column near the 150 MW point. These curves have been overlaid in FIG. 11 to emphasize the fact that the presence of DNA-PK_{cs} does not alter the mobility of the Ku-³H-IP₆ complex along the gel filtration column. This suggests that 1) Ku binds ³H-IP₆ and that 2) DNA-PK_{cs} does not interact with either the ³H-IP₆ or the Ku-IP₆ complex. It is believed that DNA-PK assembles on DNA and that in the absence of DNA that Ku and DNA-PK_{cs}, are separate in solution. The addition of DNA to the Ku-³H-IP₆ complex results in a shift in the elution profile from the gel filtration column—the peak is broader (possibly owing to the mobility of the DNA) and has an earlier start point. This data suggests that a Ku-³H-IP₆-DNA complex can be formed in solution. Finally, the addition of DNA-PK_{cs}, to the Ku-IP₆DNA complex results in an additional increase in mobility along the gel filtration column. The species appears very large—near 670 kD—which is consistent with it containing Ku 70/80, ³H-IP₆, DNA-PK_{cs} and DNA.

Gel filtration was carried out on DNA-PK+³H-IP₆ in the presence or absence of DNA to generate the curves in FIG. 12. Western blot analysis of these fractions was performed to detect the presence of DNA-PK_{cs} or Ku 70/80 in column fractions. As shown in FIG. 11 the peak of ³H-IP₆ is coincident with the peak of Ku 70/80, but not with the peak of DNA-PK_{cs}.

However, in the presence of DNA all 3 peaks (³H-IP₆, Ku 70/80 and DNA-PK_{cs}) are coincident which is consistent with a supercomplex containing Ku 70/80, ³H-IP₆, DNA-PK_{cs} and DNA.

The specificity of IP₆ binding by Ku was examined by competition analysis (FIG. 13) using IP₃, IP₆ and IP₇. As shown in FIG. 13, one molar equivalent of IP₆ or IP₇ resulted in an approximate 50% loss in ³H-IP₆ binding, while a 3-fold molar excess of IP₃ had no effect. These data indicate that Ku specifically recognizes IP₆. In addition, that IP₆ and IP₇ are recognized equally by Ku indicates that the binding of an inositol polyphosphate (IP) by Ku is not the source of the 10-fold increase in NHEJ stimulation observed with IP₇. These findings may be extended to postulate that while IP₆ is bound by Ku, that the stimulatory effect of IP₆ on NHEJ probably results from additional interactions that may be regulated by formation of the IP₆-Ku complex.

### EXAMPLE 5

### Spin Columns Used to Examine Inositide Phosphate Binding Species

While gel filtration can provide a wealth of information regarding complex formation it is time-consuming and limited to the examination of single samples. To facilitate the study of Ku-IP interactions a spin-column method based on the work of Kavran et al (1998) J. Biol. Chem. 273, 30497-30508 was developed. The high charge-to-mass ratio of IP₆ gives it a large apparent molecular size in aqueous solution. As such, this method uses the Bio-Gel P-30 resin (BioRad) which has a molecular exclusion size of 40 kD. The results of specificity trials using this spin column method are presented in FIG. 14. Clearly, the level of specificity observed using these spin-columns is identical to that presented in FIGS. 9 and 13. This method will facilitate the examination of IP binding species.

1. A method of stimulating non-homologous end-joining (NHEJ) of DNA the method comprising performing NHEJ of DNA in the presence of inositol hexalisphosphate (IP₆) or other stimulatory inositol phosphate.

2. An assay of non-homologous end-joining (NHEJ) of DNA wherein the assay comprises inositol hexakisphosphate (IP₆) or other stimulatory inositol phosphate.

3. A method according to claim 1 or assay according to claim 2 wherein the IP₆ or other stimulatory inositol phosphate is exogenous IP₆ or other stimulatory inositol phosphate.

4. A method or assay according to any one of the preceding claims wherein the NHEJ is performed in vitro.

5. A method or assay according to any one of the preceding claims wherein the NHEJ of DNA is performed in a NHEJ reaction mixture which includes DNA-dependent protein kinase, XRCC4, DNA ligase IV and a suitable DNA substrate.

6. Use of IP₆ or other stimulatory inositol phosphate for stimulating non-homologous end-joining of DNA.

7. A kit of parts comprising IP₆ or other stimulatory inositol phosphate and one or more of a DNA-dependent protein kinase, XRCC4, DNA ligase IV and a suitable DNA substrate.

8. A kit of parts comprising inositol hexakisphosphate (IP₆) or other stimulatory inositol phosphate and a host cell expressing one or more of a DNA-dependent protein kinase, XRCC4 and DNA ligase IV.

9. A kit of parts according to claim 8 wherein one or more of a DNA-dependent protein kinase, XRCC4 and DNA ligase IV are expressed from a recombinant nucleic acid molecule.

10. An assay of a protein kinase wherein the assay comprises inositol hexakisphosphate (IP₆) or other stimulatory inositol phosphate.

11. A kit of parts comprising a protein kinase and inositol hexakisphosphate (IP₆) or other stimulatory inositol phosphate.

12. A kit of parts according to claim 11 wherein the protein kinase is expressed from a recombinant nucleic acid molecule.

13. An assay according to claim 10 or a kit of parts according to claim 11 further comprising a substrate for said protein kinase.

14. A kit of parts comprising inositol hexalisphosphate (IP₆) or other stimulatory inositol phosphate and a host cell expressing a protein kinase.

15. An assay according to claim 10 or a kit of parts according to any one of claims 11 to 14 wherein the protein kinase is a protein kinase which has a domain with similarity to the catalytic domain of phosphatidylinositol 3-kinase.

16. An assay or kit of parts according to claim 15 wherein the protein kinase is any one of a DNA-dependent protein kinase, ATR, ATM, FRAP, or the Saccharomyces cerevisiae gene products Tel1p, Mec1p, Tor1p or Tor2p, or the Schizosaccharomyces pombe gene product Rad3.

17. A method of identifying a compound which modulates or mimics the effect of inositol hexakisphosphate (IP₆) or other stimulatory inositol phosphate in stimulating non-homologous end-joining (NHEJ) of DNA the method comprising performing NHEJ of DNA in the presence of inositol hexakisphosphate (IP₆) or other stimulatory inositol phosphate and determining the effect of a test compound on the NHEJ of DNA.

18. A method according to claim 17 wherein the NHEJ of DNA is performed in vitro.

19. A method according to claim 17 or 18 wherein the NHEJ of DNA is performed in a NHEJ reaction mixture which includes DNA-dependent protein kinase, XRCC4, DNA ligase IV and a suitable DNA substrate.

20. A method of identifying a compound which modulates or mimics the effect of inositol hexakisphosphate (IP₆) or other stimulatory inositol phosphate in stimulating non-homologous end-joining (NHEJ) of DNA the method comprising determining, in the presence of inositol hexakisphosphate (IP₆) or other stimulatory inositol phosphate, the effect of a test compound on the interactions between the components in the NHEJ reaction mixture.

21. A method according to claim 20 wherein the components of the NHEJ reaction are a DNA-dependent protein kinase (or a component thereof such as the Ku 70/80 heterodimer or a subunit thereof), XRCC4, DNA ligase IV, a suitable DNA substrate, ATP and Mg²⁺.

22. A method of identifying a compound which modulates the non-homologous end-joining of DNA, the method comprising determining the effect of an inositol phosphate or derivative thereof on non-homologous end-joining of DNA.

23. A method of identifying a compound which modulates or mimics the effect of inositol hexakisphosphate (IP₆) or other stimulatory inositol phosphate on a protein kinase the method comprising determining, in the presence of IP₆ or other stimulatory inositol phosphate, the effect of a test compound on the catalytic activity of the protein kinase or on the ability of the protein kinase to interact with another component.

24. A method of identifying a compound which modulates the binding of IP₆ or other stimulatory inositol phosphate to a protein kinase, the method comprising determining whether a test compound reduces or increases the binding of IP₆ or other stimulatory inositol phosphate to the said protein kinase or a subunit thereof.

25. A method according to claim 23 or 24 wherein the protein kinase is a protein kinase which has a domain with similarity to the catalytic domain of phosphatidylinositol 3-kinase.

26. A method according to claim 25 wherein the protein kinase is any one of a DNA-dependent protein kinase, ATR, ATM, FRAP, or the Saccharomyces cerevisiae gene products Tel1p, Mec1p, Tor1p or Tor2p, or the Schizosaccharomyces pombe gene product Rad3.

27. A method according to any one of claims 23 to 26 wherein the protein kinase is a DNA-dependent protein kinase.

28. A method according to claim 23 wherein the effect of a test compound on the interaction between the catalytic subunit of a DNA-dependent protein kinase and any one of Ku70, Ku80, DNA ligase IV, XRCC4 or a suitable DNA substrate thereof is determined.

29. A method according to claim 24 wherein the subunit is the Ku 70/80 heterodimer of DNA-PK or the Ku70 subunit thereof or the Ku80 subunit thereof.

30. A method of identifying a compound which modulates the binding of IP₆ or other stimulatory inositol phosphate to the Ku 70/80 heterodimer of DNA-PK or the Ku70 subunit thereof or the Ku80 subunit thereof, the method comprising determining whether a test compound reduces or increase the binding of IP₆ or other stimulatory inositol phosphate to the said Ku 70/Ku 80 heterodimer or the Ku70 subunit thereof or the Ku80 subunit thereof.

31. A method of identifying a compound which modulates the binding of IP₆ or other stimulatory inositol phosphate to XRCC4 or DNA ligase IV, the method comprising determining whether a test compound reduces or increases the binding of IP₆ or other stimulatory inositol phosphate to the said XRCC4 or DNA ligase IV.

32. A method according to any one of claims 17 to 31 wherein the test compound is an inositol derivative.

33. A method according to any one of claims 17 to 31 wherein the test compound is a phosphoinositide or an analogue of inositol hexakisphosphate (IP₆) or analogue of another stimulatory inositol phosphate.

34. A method according to any one of claim 17 to 33 wherein a test compound which mimics or modulates the effect of inositol hexakisphosphate (IP₆) or other stimulatory inositol phosphate is selected for further study.

35. A method according to any one of claims 17 to 22 and 28 to 31 wherein the method is used for identifying compounds which may be useful in developing agents for treating cancer, augmenting cancer radiotherapy and/or chemotherapy regimes, improving gene therapy regimes, enhancing homologous recombination, treating retroviral infections, and modulating the immune system.

36. A method according to any one of claims 17 to 22 and 28 to 31 comprising the further steps of selecting a test compound which mimics or modulates the effect of inositol hexakisphosphate (IP₆) or other stimulatory inositol phosphate and testing it in a further screen for its suitability as an agent for treating cancer, augmenting cancer radiotherapy and/or chemotherapy regimes, improving gene therapy regimes, enhancing homologous recombination, treating retroviral infections, or modulating the immune system.

37. A method according to any one of claims 23 to 28 wherein the method is used for identifying compounds which may be useful in developing agents for modulating protein kinase activity or interactions.

38. A method according to claim 37 wherein the protein kinase is any one of DNA-PK, ATM, ATR or FRAP and the method is used for identifying compounds which modulate cell cycle checkpoint control.

39. A compound identifiable by the method of any one of claims 17 to 38.

40. A compound identified by the method of any one of claims 17 to 38.

41. A compound according to claim 39 or 40 for use in medicine.

42. A method of reducing non-homologous end-joining (NHEJ) of DNA the method comprising reducing the amount of, or inhibiting the stimulatory effect of, inositol hexakisphosphate (IP₆) or other stimulatory inositol phosphate in a NHEJ reaction.

43. A method of enhancing non-homologous end-joining (NHEJ) of DNA the method comprising increasing the amount of, or enhancing or mimicking the stimulatory effect of, inositol hexakisphosphate (IP₆) or other stimulatory inositol phosphate in a NHEJ reaction.

44. A method according to claim 42 wherein the reduction of NHEJ is in a cell.

45. A method according to claim 44 wherein the cell is in a human or animal in need of reduction in NHEJ of DNA.

46. A method according to claim 45 for treating cancer, augmenting cancer radiotherapy and/or chemotherapy regimes, improving gene therapy regimes, enhancing homologous recombination, treating retroviral infections, or modulating the immune system.

47. A method according to claim 45 wherein the enhancement of NHEJ is in a cell.

48. A method according to claim 47 wherein the cell is in a human or animal in need of enhancement in NHEJ of DNA.

49. A method according to claim 48 for treating patients who are immunocompromised or susceptible to cancer due to impaired checkpoint cell cycle control.

50. A method of modulating the activity or interaction of a protein kinase the method comprising changing the amount of inositol hexakisphosphate (IP₆) or other stimulatory inositol phosphate present with the protein kinase, or inhibiting or enhancing the effect of IP₆ or other stimulatory inositol phosphate on the protein kinase.

51. A method according to claim 46 wherein the protein kinase is a protein kinase which has a domain with similarity to the catalytic domain of phosphatidylinositol 3-kinase.

52. A method according to claim 50 wherein the protein kinase is in a cell.

53. A method according to claim 52 wherein the cell is in a human or animal in need of modulation of protein kinase activity or interaction.

54. A method according to claim 53 wherein the protein kinase is any one of DNA-PK, ATM, ATR or FRAP and the method is for modulating cell cycle checkpoint control.

55. A method of determining whether an individual has or is predisposed to a defect in DNA repair or cell cycle checkpoint control, the method comprising the steps of (1) obtaining a sample from the patient, (2) determining the concentration of, or subcellular localisation of, inositol hexakisphosphate (IP₆) or other stimulatory inositol phosphate in the sample, and (3) comparing the result with a standard.

56. Any novel screening assay or method of modulating non-homologous end-joining of DNA as herein described.
